(19) United States
(12) Patent Application Publication
(10) Pub. No.: US 2011/0171649 A1
(43) Pub. Date: Jul. 14, 2011

(54) DETECTION OF NUCLEIC ACIDS BY OLIGONUCLEOTIDE PROBES CLEAVED IN PRESENCE OF ENDONUCLEASE V

(76) Inventor: Igor Kutyavin, Woodinville, WA (US)

(21) Appl. No.: 13/063,185

(22) PCT Filed: Sep. 9, 2009
(86) PCT No.: PCT/US2009/056421

§ 371 Date: Mar. 9, 2011

Related U.S. Application Data
(60) Provisional application No. 61/095,870, filed on Sep. 10, 2008.

(51) Int. Cl. C12Q 1/68

(52) U.S. Cl. 435/6.11

(57) ABSTRACT

Particular aspects provide nucleic acid detection methods comprising contacting a test sample having a nucleic acid target sequence with at least one endo-V-cleavable oligonucleotide probe complementary to the target sequence in the presence of an endonuclease V, incubating the reaction mixture under conditions suitable to support hybridization of the endo-V-cleavable oligonucleotide probe with the target nucleic acid and endonuclease V-mediated cleavage of the target-hybridized probe, and detecting at least one endonuclease V-mediated cleavage product of the target-hybridized probe wherein the presence of the cleavage products is indicative of the presence of the target nucleic acid sequence in the sample. Particular aspects comprise amplification of the target nucleic acid sequence before and/or during the incubating and/or detecting, wherein detecting is post-amplification and/or real-time. Additional aspects provide suitable kits. Further aspects comprise use of at least one nick-directing modification or other structural modification of the at least one endo-V-cleavable oligonucleotide probe.

### CROSS-REFERENCE TO RELATED APPLICATIONS

This application claims the benefit of priority to U.S. Provisional Patent Application Ser. No. 61/095,870, which is incorporated herein by reference in its entirety.

### STATEMENT REGARDING FEDERALLY-SPONSORED RESEARCH

This invention was made with government support under Contract 1R43GM088934-01 awarded by the National Institutes of Health. The Government has certain rights in the invention

### FIELD OF THE INVENTION

Aspects of the present invention relate generally to compositions and methods for detection nucleic acids (e.g., DNA and RNA), and more particularly to novel compositions and methods for detection of a target nucleic acid sequence, comprising incubating, in the presence of an endonuclease V activity, the target nucleic acid sequence in a suitable reaction mixture and conditions with at least one endo-V-cleavable oligonucleotide probe that is complementary to the target nucleic acid sequence to provide for endonuclease V-mediated cleavage of target-hybridized probe, and detecting at least one cleavage product. Particular aspects of the methods, comprise amplification of the target nucleic acid sequence during said incubation and/or detection, wherein detection takes place either after (post-amplification methods) or during (real-time methods) target sequence amplification.

### BACKGROUND

Early recognition of pathogens and genetic diseases, and susceptibility and/or predisposition thereto is vitally important in healthcare and, at least in part, depends on the ability to detect nucleic acids with accuracy and sensitivity. Not surprisingly, DNA and RNA detection methods are now routinely used for forensic, paternity, military, environmental and other testing applications. Although some highly sensitive technologies for direct nucleic acid detection are currently under development, amplification of targeted sequences is an important component of many DNA detection systems today. The polymerase chain reaction (PCR) is by far the most widely used approach for increasing the concentration of a segment of target sequence in a mixture of DNA without cloning or purification (see, e.g., U.S. Pat. Nos. 4,683,195 and 4,683,202 to Mullis K. B., 1987), and is a commonly used for DNA amplification to increase the concentration of target polynucleotides to a detectable level (e.g., starting from as little as a single copy of DNA or RNA). Fluorimetric detection of PCR products has significantly simplified readout and made possible real-time techniques that allow amplification to be monitored continuously (see, e.g., Higuchi, R. et al, 1992 and 1993). Fluorescent markers can be detected at nanomolar concentrations, well within the range of productivity of PCR and many other amplification techniques.

Fluorescence-based systems have been further improved by labeled-oligonucleotide probe detection. These detection techniques are based on the principle of complementarity, where an oligonucleotide sequence can, for example, be chosen to form a perfect match duplex with any predetermined site of a polynucleotide sequence of interest. This complementary duplex can then be detected indicating the presence of the targeted nucleic acid in the reaction mixture. Fluorescent probes are oligonucleotides designed to bind exclusively to a target polynucleotide. In the most commonly used methods, these probes are synthesized with both a reporter fluorescent dye and a quencher dye, and the target detection employs the Förster Resonance Energy Transfer (FRET) effect (Förster T., 1965). Regardless of design, all FRET probes function by one of two strategies. In first strategy the magnitude of FRET is based on a change of distance between the donor and acceptor dyes as result of sequence-specific hybridization between a target nucleic acid and a fluorescent oligonucleotide probe_(Hybridization-triggered FRET probes). The second strategy is based on the probe cleavage.

Cleavable FRET probes. The best strategy to abolish FRET is based on cleavage of the oligonucleotide probes upon their binding to target nucleic acids. When the probe cleavage takes place somewhere between the conjugated FRET dyes, this permanently and irreversibly disrupts FRET. In this strategy originally disclosed in Duck P. et al, 1989, a cleavable probe binds to a target nucleic acid in a sequence-specific fashion and is then recognized and cleaved by an enzyme. Two provisions have to be met in order to develop a nucleic acid detection assay based on this concept. First, the cleavage reaction must be duplex-specific so the probe gets cleaved when it is hybridized to the target. Second, the enzyme must recognize and preferentially cleave the probe strand. If the hybridized probe is cleaved internally, the cleavage products form weaker hybrids than the original, intact probe and dissociate from the target strand, leaving that strand available for additional rounds of the cleavage reaction (e.g., target cycling, or probe cycling at a target sequence). The fundamental principal of Cycling Probe Technologies (CPT) is that more than one probe can be cleaved per target molecule sequence. A number of duplex-specific endonucleases and corresponding probe designs that satisfy both major requirements of CPT have been reported.

For example, one approach is based on use of chimeric DNA-RNA probes that are cleaved by RNAse H upon the binding to target DNA (see, e.g. Duck P. et al, 1989; Fong W. et al, 2000). These DNA probes are designed to have at least 4-5 ribonucleotides in the middle of the oligonucleotide chain (e.g., Harvey J. J. et al, 2004). RNAse H cleaves only the RNA portion of the hybridized probe and the target polynucleotide is recycled to hybridize to another uncleaved probe molecule. Under appropriate conditions, this leads to a cycling of the probe cleavage reaction.

By contrast, TaqMan™ technology was developed as a real-time PCR-based detection method and utilizes the 5′-3′ exonuclease activity of Thermus aquaticus (Taq) polymerase (see, e.g., U.S. Pat. Nos. 5,210,015 and 5,487,972 to Gelfand D. H. et al, 1993 and 1996). A dual labeled FRET probe is designed to anneal to a target sequence located between two PCR primer binding sites. During strand elongation, Taq polymerase cleaves the probe that is hybridized downstream from a primer site, releasing the reporter dye from the quencher. Nonetheless, however, TaqMan™ technology is not a CPT method of nucleic acid detection.

Another example of the CPT concept is the art-recognized INVADER™ assay that utilizes the ‘flap’ or 5′-endonuclease activity of certain polymerases to cleave two partially overlapping oligonucleotides upon their binding to target DNA (see, e.g., U.S. Pat. Nos. 5,691,142 and 5,837,450 to Dahlberg, J. E. et al, 1997 and 1998). In the most advanced forms, the INVADER™ assay consists of two consecutive cycling cleavage reactions (e.g., Hall J. G. et al, 1999). The enzyme used to provide the cleavage reaction is CLEAVASE™, a DNA polymerase with substantially reduced or completely abolished synthetic capabilities. In particular embodiments, the INVADER™ assay is an efficient signal amplification system which may not require any prior target DNA amplification.

Another approach for achieving target-specific probe cleavage is based on the substrate specificity of Endonuclease IV from E. coli, an AP endonuclease that initiates repair of abasic sites and other related lesions in DNA (Kutyavin I. V. et al, 2006 and 2007). In this detection method, a FRET probe and enhancer collectively form a substrate for the AP endonuclease that simulates a partially degraded abasic site. The enzyme recognizes this artificial substrate and “clips” the 3′-tail of the probe thereby releasing the reporter dye and disrupting FRET. In particular embodiments, this reaction can be performed in a cycling mode where a high yield of cleaved probe is achieved at nanomolar or even sub-nanomolar concentrations of DNA target.

All of these art-recognized methods based on target-specific cleavage of oligonucleotide probes are not optimal in particular respects, and are further limited in applicability with respect to detection of nucleic acids. For example, the INVADER™ assay strategy can be problematic for development of real-time assays based known isothermal amplification reactions, particularly those based on strand-displacement mechanisms, due to interference of 5′-nuclease with the amplification process. Chimeric DNA-RNA probes are challenging to manufacture, and these probes have limited shelf-life because of the relative instability of the RNA segment.

There is therefore, a pronounced need in the art for more efficient, versatile and rapid methods of nucleic acid detection, including methods that are not limited by the sequence of target nucleic acids of interest, multiplexing capabilities, choice of amplification technology or requirements for post-amplification detection, sensitivity (i.e., minimum target load) and selectivity of amplification, and other factors and parameters that define the scope of the method's applicability in science and technology.

### SUMMARY OF EXEMPLARY ASPECTS OF THE INVENTION

Particular aspects of the present invention provide methods for detecting a target nucleic acid in a sample, comprising: contacting the sample with at least one endo-V-cleavable oligonucleotide probe that is substantially complementary to the target nucleic acid in a media incorporating an endonuclease V; incubating the reaction mixture under conditions suitable to support hybridization of the endo-V-cleavable oligonucleotide probe with the target nucleic acid and cleavage of the hybridized probe by the endonuclease V; and detecting at least one of the cleavage products, wherein the presence of the cleavage products is indicative of the presence of the target nucleic acid in the sample.

In certain aspects, the target nucleic acid is single-stranded, or the target nucleic acid is double-stranded, and wherein prior to, or during, the amplification reaction the double-stranded target nucleic acid is rendered single-stranded. In particular embodiments, the target nucleic acid is DNA. In certain aspects, the target nucleic acid is RNA, or at least one DNA copy of the RNA is synthesized using a reverse transcriptase prior to amplifying the amplifiable target DNA sequence.

In certain preferred aspects of the invention, the Endonuclease V is a mutant Endonuclease V that, compared to the native enzyme, has improved properties for practicing the methods of the invention. The properties that may be improved by the Endo V enzyme mutation include but not limited to: (i) increased efficiency and cycling capabilities of the probe cleavage reaction, (ii) preferential cleavage of the probe strand when it is hybridized to the target with minimal or no cleavage of the target strand, and (iii) preferential cleavage of the probe incorporating nick-directing (ND) modification when it is hybridized to the target compare to the cleavage of the unhybridized probe.

In particular aspects of the methods, the endo-V-cleavable oligonucleotide probe incorporates one or more ND modifications. In other aspects, the ND modifications are selected from deoxyinosine, deoxyxanthosine, deoxyuridine, abasic nucleotide and mismatched nucleotide. The main activity of Endonucleases V is to recognize and cleave DNA duplexes incorporating deoxyinosine where the enzymes express maximum cleavage activity. In preferred aspects of the methods, the ND modification is deoxyinosine. The nick-directing modifications are preferably located at the 5′-end of the endo-V-cleavable oligonucleotide probe. Endonucleases V recognize deoxyinosine and cleave the corresponding strand in both single and double-stranded DNA but exhibit approximately 4-fold preference for double-stranded over single-stranded DNA. However, this cleavage preference can be considerably improved up to 15-fold when deoxyinosine is located at the terminal 5′-position, and the result underscores the importance of the deoxyinosine location within the oligonucleotide probe. When the ND modification is deoxyinosine, it is preferably located at the third, more preferably at the second, and even more preferably at the first nucleotide position with respect to the 5′-end of the endo-V-cleavable oligonucleotide probe. The observed considerable improvement in the probe cleavage rate and the duplex-specificity of cleavage (up to 15-fold) were unexpected as described in detail herein below.

In certain aspects, the endo-V-cleavable oligonucleotide probe incorporates structural modifications other than the ND modifications. In a preferred embodiment, these structural modifications are duplex-stabilizing modifications. In one embodiment, the duplex-stabilizing modifications are modified nucleotides. In another embodiment, duplex-stabilizing modifications comprise a minor groove binders (MGB) and intercalators. In preferred embodiment, the other structural modifications are introduced into the probe to improve efficiency and/or cycling capabilities of the probe cleavage reaction; preferential cleavage of the probe strand when it is hybridized to the target; preferential cleavage of the probe when it is hybridized to the target compare to the cleavage of the unhybridized probe and/or combination thereof.

In particular aspects, the other structural modifications comprise a detectable label. In one embodiment, the detectable label is a fluorescent label. In preferred embodiments, the fluorescent label comprises two dyes that are in FRET interaction, and wherein cleavage of the endo-V-cleavable oligonucleotide probe takes place between the conjugated dyes, disrupting FRET and resulting in detectable signal.

In preferred embodiments of the invention, the detected target nucleic acid is amplified before and/or during the detection reaction employing one of the amplification reactions presently described in the Art. In another preferred embodiment of the invention, the target nucleic acid is amplified and detected in real-time. In real-time methods of the invention, all amplification components such as, e.g. oligonucleotide primers, DNA polymerase, other enzymes, etc., and detection components such as the endo-V-cleavable probes and Endonuclease V are present in the same reaction mixture. This permits a target nucleic acid to be measured as the amplification reaction progresses, decreasing the number of subsequent handling steps required for the detection of amplified material. In particular preferred embodiments, the target nucleic acid is amplified and detected in real-time using Accelerated Cascade Amplification. In these particular methods, Endonuclease V works to cleave primer and probes during both the amplification and detection reactions, reducing the need for adjustment and optimization of the real-time reaction composition, conditions and reaction protocol.

In particular embodiments of the invention, detection of the target nucleic acid is performed to measure amount of the target nucleic acid in or from the sample. In other embodiments, more than one target nucleic acid is detected providing at least one endo-V-cleavable oligonucleotide probe for each target nucleic acid detected (multiplex detection). In such aspects, the methods of the invention can be used to detect target polymorphic variations such as, for example, sequence insertions, deletions, and other polymorphic variations as small as single nucleotide. In particular preferred aspects, the cleavage of the hybridized probe by the endonuclease V is performed in a cycling mode providing more than one probe cleavage per a target nucleic acid molecule during the detection.

Additional aspects provide nucleic acid detection kits for practicing the inventive methods. In further aspects, at least one of the target nucleic acid, the endo-V-cleavable oligonucleotide probe, and the endonuclease V is immobilized.

### BRIEF DESCRIPTION OF THE DRAWINGS

FIG. 1 shows art-recognized structures of mutant nucleosides that appear in DNA as result of base deamination. These mutant nucleosides may be used in methods of the invention as nick-directing modifications.

FIG. 2 shows, according to particular exemplary aspects of the present invention, the mechanism of detection of a target nucleic acid using an endo-V-cleavable probe in presence of Endonuclease V. Abbreviation N means any nucleotide. In stage A, the probe incorporating a nick-directing modification D hybridizes to a complementary region of the target nucleic acid forming a duplex. Endonuclease V recognizes this duplex in stage B and cleaves the probe strand at the phosphodiester bond shown by arrow (cleavage site). The products of the probe cleavage dissociate in stage C. These products are detected providing correspondingly detection of the nucleic acid of interest in a sample. Various preferred aspects of design of the endo-V-cleavable probes and, in particular, the nick-directing modifications including its nature and location within the probes are discussed in the text. Certain examples of the nick-directing modifications are shown in FIG. 1. In preferred embodiments, the nick-directing modification is deoxyinosine located at the first, second or third position from the 5′-end of the probe as exemplified in FIG. 3.

FIG. 3 shows, according to particular exemplary aspects of the present invention, a 19-mer target oligonucleotide (SEQ ID NO:4 (TAAAACGGCACCGGAATCG)) which was detected in reaction with three 11-12-mer endo-V-cleavable FRET probes incorporating deoxyinosine as a nick-directing modification located at the first (SEQ ID NO:1 (NTGGCCTTAGC)), second (SEQ ID NO:2 (CNTGGCCTTAGC)) or third (SEQ ID NO:3 (CCNTGGCCTTAG)) position from the 5′-end of the probes, respectively. Abbreviation of deoxyinosine is N in the sequence listing and I in the drawings, whereas F is 6-fluorescein and Q is BHQ1 quencher from Biosearch Technologies, Inc. The mechanism of the target detection is shown for the probe incorporating deoxyinosine at the 5′-terminal position. The example shown herein is based on fluorescence detection. Cleavage of the hybridized probe, which results in a detectable cleavage product (SEQ ID NO:5 (GGCCTTAGC)) permanently and irreversibly disrupts the FRET interaction between the reporter (F) and quencher (Q) dyes and results in a detectable fluorescent signal. Results of the fluorescence monitoring in reactions are shown in FIG. 4.

FIG. 4 shows, according to particular exemplary aspects of the present invention, results of fluorescence monitoring of the cleavage of three deoxyinosine-incorporating FRET probes (SEQ ID NO:1 (NTGGCCTTAGC), SEQ ID NO:2 (CNTGGCCTTAGC), and SEQ ID NO:3 (CCNTGGCCTTAG)) by Endonuclease V of Escherichia coli in presence (hollow marks) and absence (black marks) of the detected 19-mer target oligonucleotide (SEQ ID NO:4 (TAAAACGGCACCGGAATCG)). Structures of the target oligonucleotide and the probes are shown in FIG. 3. The fluorescence curves are identified for each probe by arrows. A detailed description of the experimental reactions and results is provided herein under working EXAMPLE 2.

### DETAILED DESCRIPTION OF EXEMPLARY EMBODIMENTS

### Definitions

Terms and symbols of biochemistry, nucleic acid chemistry, molecular biology and molecular genetics used herein follow those of standard treaties and texts in the field (e.g., Sambrook J. et al, 1989; Kornberg A. and Baker T., 1992; Gait M. J., ed., 1984; Lehninger A. L., 1975; Eckstein F., ed., 1991, and the like). To facilitate understanding of particular exemplary aspects of the invention, a number of terms are discussed below.

In particular aspects, “sample” refers to any substance containing or presumed to contain a nucleic acid of interest. The term “sample” thus includes but is not limited to a sample of nucleic acid, cell, organism, tissue, fluid, for example, spinal fluid or lymph fluid, or substance including but not limited to, for example, plasma, serum, urine, tears, stool, respiratory and genitourinary tracts, saliva, semen, fragments of different organs, tissue, blood cells, samples of in vitro cell cultures, isolates from natural sources such as drinking water, microbial specimens, and objects or specimens that have been suspected to contain nucleic acid molecules.

In particular aspects, “target nucleic acid” or “nucleic acid of interest” refers to a nucleic acid or a fragment of nucleic that is to be amplified and/or detected using methods of the present invention. Nucleic acids of interest can be of any size and sequence; e.g. as big as genomic DNA. Preferably, the nucleic acid is of a size that provides for detection and/or amplification thereof. Two or more target nucleic acids can be fragments or portions of the same nucleic acid molecule. As used herein, target nucleic acids are different if they differ in nucleotide sequence by at least one nucleotide. In this aspect, the invention may be used to detect “polymorphic variations” wherein, for example, two nucleic acids of interest have significant degree of identity in the sequence but differ by only a few nucleotides (e.g. insertions, deletions) or by a single nucleotide, or single nucleotide polymorphism (SNP). Target nucleic acids can be single-stranded or double-stranded. When nucleic acid of interest is double-stranded or presumed to be double-stranded, the term “target nucleic acid” refers to a specific sequence in either strand of double-stranded nucleic acid. Therefore the full complement to any single stranded nucleic acid of interest is treated herein as the same (or complementary) target nucleic acid. In certain aspects, target nucleic acids of the invention comprise polynucleotides comprising natural and/or modified nucleotides, if presence of these structural modifications is beneficial for the detection by the methods of the invention, e.g. duplex-stabilizing base-modified nucleotide to enhance hybridization properties of the endo-V-cleavable probes.

In particular aspects, “amplification” and “amplifying” target nucleic acids, in general, refers to a procedure wherein multiple copies of the nucleic acid of interest are generated in the form of DNA copies.

In particular aspects, “amplicon” or “amplification product” refers to a primer extension product or products of amplification that may be a population of polynucleotides, single- or double-stranded, that are replicated from either strand or from one or more nucleic acids of interest. Regardless of the originating target nucleic acid strand and the amplicons state, e.g. double- or single-stranded, all amplicons which are usually homologous are treated herein as amplification products of the same target nucleic acid including the products of incomplete extension.

In particular aspects, the terms “complementary” or “complementarity” are used herein in reference to the polynucleotides base-pairing rules. Double-stranded DNA, for example, consists of base pairs wherein, for example, G forms a three hydrogen bond couple, or pairs with C, and A forms a two hydrogen bond complex, or pairs with T, and it is regarded that G is complementary to C, and A is complementary to T. In this sense, for example, an oligonucleotide 5′-GATTTC-3′ is complementary to the sequence 3′-CTAAAG-5′. Complementarity may be “partial” or “complete.” For example, as referred to herein the phrase “a probe that is complementary to the target nucleic acid”, the term “complementary” incorporates both partial and complete complementarity. In partial complementarity, only some of the nucleic acids bases are matched according to the base pairing rules. The degree of complementarity between nucleic acid strands has significant effects on the strength of hybridization between nucleic acids. This is particularly important in performing amplification and detection reactions that depend upon nucleic acids binding. The terms may also be used in reference to individual nucleotides and oligonucleotide sequences within the context of polynucleotides. As used herein, the terms “complementary” or “complementarity” refer to the most common type of complementarity in nucleic acids, namely Watson-Crick base pairing as described above, although the probes, oligonucleotide components and amplification products of the invention may participate, including an intelligent design, in other types of “non-canonical” pairings like Hoogsteen, wobble and G-T mismatch pairing.

In particular aspects, the term “homology” and “homologous” refers to a degree of identity between nucleic acids. There may be partial homology or complete homology.

In particular aspects, the term “secondary structure” refers to an intermolecular complex formation of one sequence in a polynucleotide with another sequence in the same polynucleotide due to complete or partial complementarity between these two sequences. Unless specified otherwise, the term “complex” means the same as “duplex” and it represents a double-stranded fragment or portion of a nucleic acid formed on the principal rules of the Watson-Crick base pairing. The terms “hairpin” structure or “stem-loop” structure may be also used herein describing elements of secondary structure and both terms refer to a double-helical region (stem) formed by base pairing between complementary sequences in a single strand RNA or DNA.

In particular aspects, “isothermal amplification” and “isothermal amplification reaction” refers to a process which generates multiple copies of a target nucleic acid, and which, unlike PCR, does not require temperature changes (temperature cycling) during the amplification, and which may rather be conducted at a relatively steady or relatively constant temperature. Reaction temperature in isothermal amplification, including in methods of the invention may fluctuate somewhat, but is not required for the purpose of amplicon strand separation as in PCR.

“PCR” is an abbreviation of term “polymerase chain reaction,” the art-recognized nucleic acid amplification technology (e.g., U.S. Pat. Nos. 4,683,195 and 4,683,202 to Mullis K. B.). The commonly used PCR protocol employs two oligonucleotide primers, one for each strand, designed such that extension of one primer provides a template for the other primer in the next PCR cycle. Generally, a PCR reaction consists of repetitions (or cycles) of (i) a denaturation step which separates the strands of a double-stranded nucleic acid, followed by (ii) an annealing step, which allows primers to anneal to positions flanking a sequence of interest, and then (iii) an extension step which extends the primers in a 5′ to 3′ direction, thereby forming a nucleic acid fragment complementary to the target sequence. Each of the above steps may be conducted at a different temperature using an automated thermocycler. The PCR cycles can be repeated as often as desired resulting in an exponential accumulation of a target DNA amplicon fragment whose termini are usually defined by the 5′-ends of the primers used. Particular temperatures, incubation times at each step and rates of change between steps depend on many factors well-known to those of ordinary skill in the art and the examples can be found in numerous published protocols (e.g., McPherson M. J. et al., 1991 and 1995) and the like). Although conditions of PCR can vary in a broad range, a double-stranded target nucleic acid is usually denatured at a temperature of >90° C., primers are annealed at a temperature in the range of about 50-75° C., and the extension is preferably performed in the 72-7° C. range. The term “PCR” encompasses derivative forms of the reaction, including but not limited to, “RT-PCR,” “real-time PCR,” “nested PCR,” “quantitative PCR,” “multiplexed PCR,” “asymmetric PCR” and the like.

The term “Accelerated Cascade Amplification” (“ACA”) refers to an isothermal amplification which is based on use of at least three nick-directing primers as described in Nelson J. R. et al, (2008) and Kutyavin I. (2009).

As used herein, the term “nuclease” refers to an enzyme which expresses a phosphomonoesterase or phosphodiesterase activity and capable of cleaving a phosphorester bond in compounds such as R′—O—P(O)(OH)₂ and R′—O—P(O)(OH)—O—R″ resulting in products R′—OH+P(O)(OH)₃ and R′—OH+P(O)(OH)₂—O—R″ (or R″—OH+P(O)(OH)₂—O—R′), respectively and wherein R′ and R″ may be moieties of any structure which are not necessarily of a nucleotide nature. The term “nucleases” incorporates both “exo” and “endo” nucleases.

As used herein, the terms “Endonuclease V” or “Endo V” encompasses nucleases that express the enzymatic activities found, for example, in Endonucleases V from Escherichia coli (Yao M. et al, 1994; Yao M. and Kow Y. W., 1994; Yao M., Kow Y. W., 1995; Yao M., Kow Y. W., 1997), Archaeoglobus fulgidus (Liu J. et al, 2000) and Thermotoga maritima (Huang J. at al, 2001 and 2002). The main enzymatic activity of Endonucleases V used in the methods of the invention is preferential hydrolysis of the second phosphodiester bond in a DNA strand at the 3′ side from a deoxyinosine position/modification. The Endonucleases V of the invention are preferably duplex specific but they may cleave nucleic acids incorporating nick-directing modifications when these nucleic acids are in single stranded state. For example, Endonucleases V are known to cleave single-stranded DNA polymers incorporating deoxyinosine and deoxyuridine modifications. The Endonucleases V of the invention are preferentially essentially free of, or express very little “general” duplex-specific or single-strand-specific nuclease activity so as not to cleave oligonucleotides unless these oligonucleotides incorporate ND modifications.

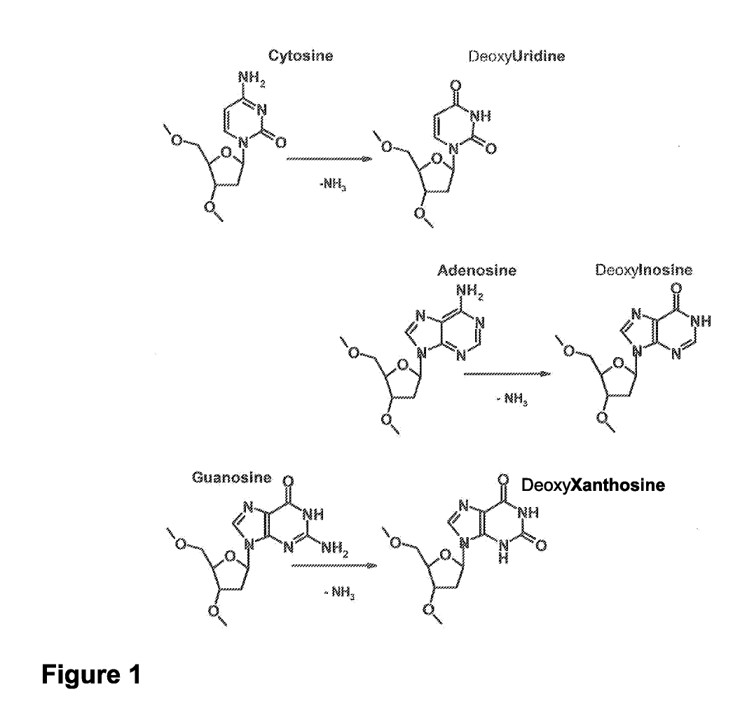

The term “nick-directing modifications” (“ND modifications”) as used herein, has very broad meaning and it refers to any approach or structural entity (modification) or combination thereof within double-stranded oligonucleotides or polynucleotides, which direct Endonuclease V to cleave preferentially that one, of the two duplexed strands, which incorporates the ND modification. Deoxyinosine modification is a preferred ND modification for design of the EndoV probes of the invention. Examples of other ND modifications that can be used to prepare the EndoV probes include deoxyxanthosine (Schouten K. A. and Weiss B., 1999; He B. et al, 2000), deoxyuridine, and abasic sites (Yao M. et al, 1994; Yao M., Kow Y. W., 1997), mismatches (Yao M. and Kow Y. W., 1994; Huang J. at al, 2001), pseudo Y structures, and small insertions/deletions (Yao M., Kow Y. W., 1996). Particular examples of nucleosides that may be used in methods of the invention as ND modifications are shown in FIG. 1. As referred herein, the phrase “the nick-directing modifications are located at the 5′-end of the endo-V-cleavable oligonucleotide probe,” generally means, for example, that with regard to an exemplarily oligonucleotide 5′-CCGTATG-3′, the modification(s) are located within the 5′-CCGT-nucleotide sequence of 5′-CCGTATG-3′.

The terms “deoxyinosine”, “deoxyxanthosine”, “deoxyuridine”, “abasic nucleotide” and “mismatched nucleotide” refer to certain ND modifications that are most commonly used to prepare the EndoV probes of the invention. Each term encompasses various derivative forms or structural modifications of the corresponding generic structures. In this aspect, for example, the term “deoxyinosine” incorporates 8-aza-7-deaza deoxyinosine (pyrazolopyrimidine analog), (2′-O, 4′-C methylene)-inosine (LNA analog) and other structural modifications whereas the term “deoxyuridine”, e.g. incorporates deoxypseudouridine; the term “abasic nucleotide” incorporates 1′,2′-dideoxyribose; and the term “mismatched nucleotide” incorporates base-modified nucleotide analogs that do not form Watson-Crick base pairing (form mismatch) with natural nucleotides (see, e.g., Petrie C. R. et al, 1998, incorporated by reference herein for its teachings on derivatives).

In certain preferred embodiments of the present invention, detection of the target nucleic acids can be performed in “real-time” or “real time.” Real time detection is possible when all detection components are available during the amplification and the reaction conditions such as temperature, buffering agents to maintain pH at a selected level, salts, co-factors, scavengers, and the like support both stages of the reaction, amplification and the detection. This permits a target nucleic acid to be measured as the amplification reaction progresses decreasing the number of subsequent handling steps required for the detection of amplified material. “Real-time detection” means an amplification reaction for which the amount of reaction product, i.e. target nucleic acid, is monitored as the reaction proceeds. Reviews of the detection chemistries for real-time amplification can be also found in Didenko V. V., 2001, Mackay I. M. et al, 2002, and Mackay J., Landt O., 2007, which are incorporated herein by reference. In preferred embodiments of the present invention, detection of nucleic acids is based on use of FRET effect and FRET probes.

In certain aspects, the amplification and detection stages of the invention may be performed separately, not in real time, when the detection stage follows the amplification. The terms “detection performed after the amplification”, “target nucleic acid is amplified before the detection reaction” and “post-amplification detection” are used herein to describe such assays.

“Multiplexed amplification” refers to an amplification reaction wherein multiple target nucleic acids are simultaneously amplified. Correspondingly, the term “multiplexed detection” refers to a detection reaction wherein multiple target nucleic acids are simultaneously detected.

“Polynucleotide” and “oligonucleotide” are used herein interchangeably and each means a linear polymer of nucleotide monomers. Polynucleotides typically range in size from a few monomeric units, e.g. 5-40, when they are usually referred to as “oligonucleotides,” to several thousand monomeric units. The exact size will depend on many factors, which in turn depends on the ultimate function or use of the oligonucleotide. The oligonucleotides may be generated in any manner, including chemical synthesis, DNA replication, reverse transcription, or a combination thereof. Whenever a polynucleotide or oligonucleotide is represented by a sequence of letters, for example, “CCGTATG,” it is understood herein, unless otherwise specified in the text, that the nucleotides are in 5′→3′ order from left to right and that “A” denotes deoxyadenosine, “C” denotes deoxycytidine, “G” denotes deoxyguanosine, and “T” denotes deoxythymidine. Usually DNA polynucleotides comprise these four deoxyribonucleosides linked by phosphodiester linkage whereas RNA comprises uridine (“U”) in place of “T” for the ribose counterparts.

The terms “natural nucleosides” and “natural nucleotides” as used herein refer to four deoxynucleosides or deoxynucleotides respectively which may be commonly found in DNAs isolated from natural sources. Natural nucleosides (nucleotides) are deoxyadenosine, deoxycytidine, deoxyguanosine, and deoxythymidine. The term also encompasses their ribose counterparts, with uridine in place of thymidine.

As used herein, the terms “unnatural nucleosides” or “modified nucleosides” refer to nucleotide analogs that are different in their structure from those natural nucleotides for DNA and RNA polymers. The same terms can be used in regard to corresponding nucleotides that also can be “unnatural” or “modified”. Some of the naturally occurring nucleic acids of interest may contain nucleosides that are structurally different from the natural nucleosides defined above, for example, DNAs of eukaryotes may incorporate 5-methyl-cytosine and tRNAs are notorious for harboring many nucleoside analogs. However, as used herein, the terms “unnatural nucleosides” or “modified nucleotides” encompasses these nucleoside modifications even though they can be found in natural sources. For example, ribothymidine and deoxyuridine are treated herein as unnatural nucleosides. Certain modified nucleotides are used in the invention as “nick-directing modifications.” Examples include but not limited to deoxyinosine and deoxyuridine.

The term “oligonucleotide component” refers to any molecule of polynucleotide nature that is required or helpful in conducting either amplification or detection reaction of the invention or both. Oligonucleotide components include but not limited to primers including, e.g. ND primers used in NDA and ACA, probes including endo-V-cleavable probes of the invention, hybridization and cleavage enhancers, effectors, etc. Oligonucleotide components can be labeled or have structural modifications of any kind.

The terms “oligonucleotide primer” and/or “primer” refer to a single-stranded DNA or RNA molecule that hybridizes to a target nucleic acid and primes enzymatic synthesis of a second nucleic acid strand in presence of a DNA polymerase. In this case, as used herein, the target nucleic acid “serves as a template” for the oligonucleotide primer.

As used herein, the term an “oligonucleotide probe” or “probe” refers to an oligonucleotide component which is used to detect nucleic acids of interest. These terms encompasses various derivative forms such as, e.g. “hybridization-triggered probe”, “fluorescent probe”, “FRET probe”, etc.

The terms “endonuclease V-cleavable probe,” “endo-V-cleavable probe” and/or “EndoV probe” refer to an oligonucleotide component which incorporates at least one ND modification and that is recognized and cleaved by Endonuclease V providing for detection of target nucleic acids according to the methods of the invention. The EndoV probes of the invention may incorporate detectable elements like labels, e.g. dye, mass tag, etc. In preferred embodiments, the EndoV probes contain two dyes which are in a FRET interaction and wherein hybridization of the probes with corresponding target nucleic acids followed by the Endo V cleavage between the conjugated dyes results in a detectable fluorescent signal. These probes may be also referred herein as “endo-V-cleavable FRET probe” and/or “EndoV FRET probe”. The EndoV probes of the invention may be “modified” or contain “structural modifications” that, for example, enhance their hybridization properties, improve the binding or cleavage specificity, etc.

Preferably, oligonucleotide probes are cleaved by nucleases only when they hybridized to target nucleic acids. In regard to art-recognized detection methods that are based on probe cleavage, this is not always possible to achieve, and the oligonucleotide probes are commonly cleaved in the absence of the target nucleic acids, although usually at lower rates than in the presence of the nucleic acids of interest. To some extent, the probes' cleavage in the absence of the target nucleic acids may also occur in certain methods of the invention. Particular techniques have been established in the Art to avoid false-positive detection in such cases and these approaches are described, e.g. in Duck P. et al, 1989; Fong W. et al, 2000; Harvey J. J. et al, 2004; Gelfand D. H. et al, 1993 and 1996; Dahlberg, J. E. et al, 1997 and 1998; Kutyavin I. V. et al, 2006 and 2007 and many other similar manuscripts which are incorporated herein by reference. For example, a “control” reaction can be performed that is otherwise identical to the detection reaction in a test sample but that does not incorporate the test sample (no-target control). If amount of cleavage products in the detection reaction exceeds the same in the control reaction, this indicates the presence of the target nucleic acid sequence in the test sample whereas equal amounts of the cleavage products in both reactions indicates the absence of the target nucleic acid sequence in the test sample. The control reactions can be also performed with known and variable amounts of the target nucleic acid and the comparison of these control reactions with the detection reaction in a test sample can be used in methods of the invention for determining the amount of the target nucleic acid in or from the sample. Preferably, the terms “detecting,” “detecting a target” and “detecting a cleavage product” incorporate performing the discussed control reactions, or correlation with a contemporaneous or historical control value.

The term “structural modifications” refers to any chemical substances such as atoms, moieties, residues, polymers, linkers or nucleotide analogs which are usually of a synthetic nature and which are not commonly present in natural nucleic acids. As used herein, the term “structural modifications” also include nucleoside or nucleotide analogs which rarely present in natural nucleic acid including but not limited to inosine (hypoxanthine), 5-bromouracil, 5-methylcytosine, 5-iodouracil, 2-aminoadenosine, 6-methyladenosine, preudouridine and the like. Certain structural modifications may be used in the invention as nick directing modifications.

“Duplex-stabilizing modifications” refer to structural modifications, the presence of which in double-stranded nucleic acids provides a duplex-stabilizing effect when compared in thermal stability, usually measured as Tm, with respective nucleic acid complexes that have no structural modification and comprised natural nucleotides. Duplex-stabilizing modifications are structural modifications that are most commonly applied in synthesis of probes and primers and represented by modified nucleotides and ‘tails’ like intercalators and minor groove binders.

“Hybridizing,” “hybridization” or “annealing” refers to a process of interaction between two or more oligo- and/or polynucleotides forming a complementary complex through base pairing which is most commonly a duplex or double stranded complex as originally described in Doty P. et al (1960). The stability of a nucleic acid duplex is measured by the melting temperature, or “Tm”. “Melting temperature” or “Tm” means the temperature at which a complementary complex of nucleic acids, usually double-stranded, becomes half dissociated into single strands. These terms are also used in describing stabilities of polynucleotide secondary structures wherein two or more fragments of the same polynucleotide interact in a complementary fashion with each other forming complexes, usually hairpin-like structures.

“Hybridization properties” of a polynucleotide means an ability of this polynucleotide or its fragment to form a sequence specific complex with another complementary polynucleotide or its fragment. “Hybridization properties” is also used herein as a general term in describing the complementary complex stability. In this aspect, “hybridization properties” are similar in use to yet another term, “melting temperature” or “Tm.” “Improved” or “enhanced hybridization properties” of a polynucleotide refers to an increase in stability of a complex of this polynucleotide with its complementary sequence due to any means including but not limited to a change in reaction conditions such as pH, salt concentration and composition, for example, an increase in magnesium ion concentration, presence of complex stabilizing agents such as intercalators or minor groove binders, etc., conjugated or not. The hybridization properties of a polynucleotide or oligonucleotide can also be altered by an increase or decrease in polynucleotide or oligonucleotide length. The cause of the hybridization property enhancement is generally defined herein in context.

The term “label” refers to any atom or molecule that can be used to provide a detectable signal and that can be attached to a nucleic acid or oligonucleotide. Labels include but are not limited to isotopes, radiolabels such as ³²P; binding moieties such as biotin; haptens such as digoxygenin; luminogenic, mass tags, phosphorescent or fluorescent moieties, fluorescent dyes alone or in combination with other dyes or moieties that can suppress or shift emission spectra by FRET effect. Labels may provide signals detectable by fluorescence, radioactivity, colorimetry, gravimetry, X-ray diffraction or absorption, magnetism, enzymatic activity, mass spectrometry, binding affinity and the like. A label may be a charged moiety or alternatively, may be charge neutral. Labels can include or consist of nucleic acid or protein sequence, so long as the sequence comprising the label is detectable. The term “FRET-labeled” refers a probe which usually incorporates two dyes that are in a FRET interaction.

“Fluorescent label” refers to a label that provides fluorescent signal. A fluorescent label is commonly a fluorescent dye, but it may be any molecule including but not limited to a macromolecule like protein, or a particle made from inorganic material like quantum dots, as described in (Robelek R. et al, 2004).

“FRET” is an abbreviation of Förster Resonance Energy Transfer effect. FRET is a distance-dependent interaction occurring between two dye molecules in which excitation is transferred from a donor to an acceptor fluorophore through dipole-dipole interaction without the emission of a photon. As a result, the donor molecule fluorescence is quenched, and the acceptor molecule becomes excited. The Efficiency of FRET depends on spectral properties, relative orientation and distance between the donor and acceptor chromophores (Förster T., 1965). As used herein, “FRET probe” refers to a fluorescent oligonucleotide which is used for detection of a nucleic acid of interest wherein detection is based on FRET effect.

A “reaction mixture” generally means a solution containing all the necessary reactants for performing an amplification or detection reaction or both.

The term “reaction vessel” refers to any kind of a container used to perform the amplification and/or detection reactions of the methods of the invention and wherein the term “reaction vessel” means any appropriate way of isolation of the reaction mixture from the environment.

As used herein, the term “kit” refers to any system for delivering materials. In the context of reaction assays, such delivery systems include elements allowing the storage, transport, or delivery of reaction components such as oligonucleotides, buffering components, additives, reaction enhancers, enzymes and the like in the appropriate containers from one location to another commonly provided with written instructions for performing the assay. Kits may include one or more enclosures or boxes containing the relevant reaction reagents and supporting materials. The kit may comprise two or more separate containers wherein each of those containers includes a portion of the total kit components. The containers may be delivered to the intended recipient together or separately.

The term “solid support” refers to any material that provides a solid structure with which another material can be attached. Such materials may include but not limited to silicon, plastic, metal, glass, ceramic surfaces, and the like. Solid supports may be of a rigid or non-rigid nature like gels, rubbers, polymers, etc. and may be any type of shape including spherical shapes like beads. Certain embodiments of the present invention have at least one of the reaction components such as, e.g. primer, EndoV probe, or target nucleic acid, whether amplified or not, immobilized on solid support at amplifying or detecting stages or both. A biological material is “immobilized” to a solid support when it is associated with the solid support through a random or non-random chemical or physical interaction. The immobilization or attachment may be through a covalent bond using specialty spacer molecule or linker group. However, the immobilization need not be covalent or permanent.

As used herein, “detection assay” or “assay” refers a reaction or chain of reactions that are performed to detect nucleic acids of interest. The assay may comprise multiple stages including amplification and detection reactions performed consequently or in real time, nucleic acid isolation and intermediate purification stages, immobilization, labeling, etc. The terms “detection assay” or “assay” encompass a variety of derivative forms of the methods of the invention, including but not limited to, a “post-amplification assay” when the detection is performed after the amplification stage, a “real time assay” when the amplification and detection are performed simultaneously, a “FRET assay” when the detection is based using FRET effect, “immobilized assay” when one of either amplification or detection oligonucleotide components or an amplification product is immobilized on solid support, and the like.

In general, the term “design” in the context of the methods and/or oligos, etc., has broad meaning and in certain respects is equivalent to the term “selection”. For example, the terms “oligonucleotide design”, “primer design”, “probe design” can mean or encompass selection of a particular, or sometimes not necessarily to a particular, oligonucleotide structure including the nucleotide sequence and structural modifications (e.g., labels, modified nucleotides, linkers, etc.). The term “system design” generally incorporates the terms “oligonucleotide design”, “primer design”, “probe design” and also refers to relative orientation and/or location of the oligonucleotide components and/or their binding sites within the target nucleic acids. In these aspects, the term “assay design” relates to the selection of any, sometimes not necessarily to a particular, methods including all reaction conditions (e.g. temperature, salt, pH, enzymes, oligonucleotide component concentrations, etc.), structural parameters (e.g. length and position of primers and probes, design of specialty sequences, etc.) and assay derivative forms (e.g. post-amplification, real time, immobilized, FRET detection schemes, etc.) chosen to amplify and/or to detect the nucleic acids of interest.

### Detection of Target Nucleic Acids by EndoV Probes Cleavable in Presence of Endonuclease V:

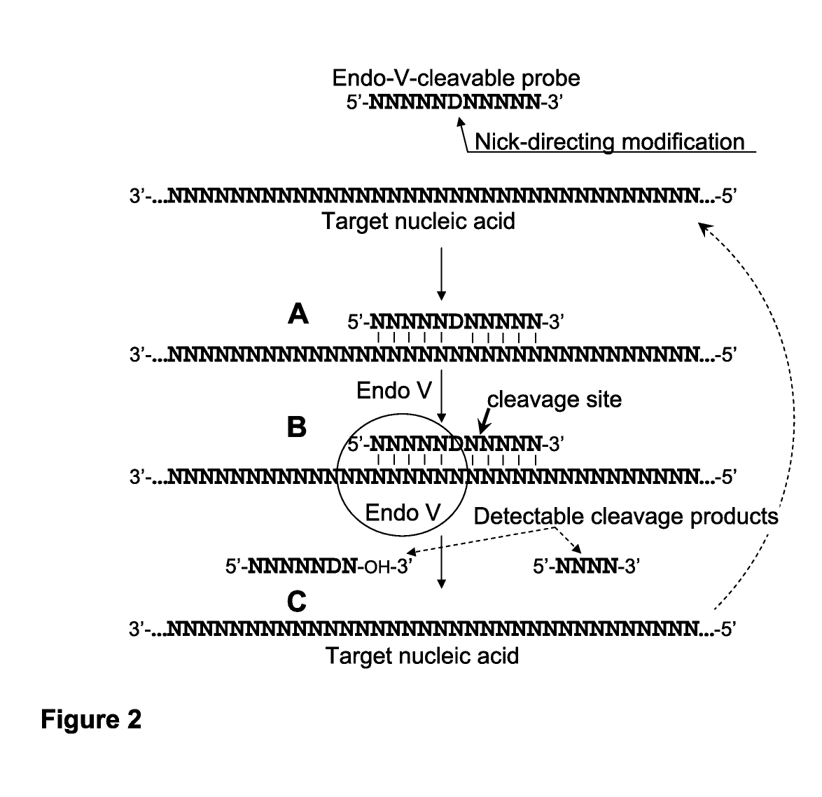

Particular aspects of the present invention provide methods for detecting a target nucleic acid in a test sample, comprising: obtaining a test sample comprising at least one target nucleic acid sequence; contacting, in the presence of an endonuclease V, the sample with at least one endo-V-cleavable oligonucleotide probe that is complementary to the target nucleic acid sequence to provide a reaction mixture; incubating the reaction mixture under conditions suitable to support hybridization of the at least one endo-V-cleavable oligonucleotide probe with the at least one target nucleic acid sequence and cleavage of the target-hybridized probe by the endonuclease V; and detecting at least one endonuclease V-mediated cleavage product of the target-hybridized probe, wherein the presence of the at least one cleavage product is indicative of the presence of the target nucleic acid sequence in the test sample. The mechanism of detection of a target nucleic acid using an EndoV probe in presence of Endonuclease V is illustrated in FIG. 2.

Target nucleic acids. Target nucleic acids, or nucleic acids of interest are preferably single-stranded. The detection reaction of the invention is initiated when an EndoV probe hybridizes to a complementary single-stranded nucleic acid forming a double-stranded substrate that is recognized and cleaved by Endonuclease V. When target nucleic acids are double-stranded, they are rendered single stranded by any physical, chemical or biological approach before applying the methods of the invention. For example, double-stranded nucleic acid can be denatured at elevated temperature, e.g. 90-95° C. The target nucleic acids may be derived from any organism or other source, including but not limited to prokaryotes, eukaryotes, plants, animals, and viruses, as well as synthetic nucleic acids. The target nucleic acids may be DNA, RNA, and/or variants thereof. Nucleic acids of interest can be isolated and purified from the sample sources before applying methods of the present invention. Preferably, the target nucleic acids are sufficiently free of proteins and any other substances interfering with detection reaction. Many methods are available for the isolation and purification of nucleic acids of interest including commercial kits and specialty instruments. For example, nucleic acids can be isolated using organic extraction with a phenol/chloroform reagent followed by ethanol precipitation (Ausubel F. M et al, eds., 1993). Solid phase adsorption method (Walsh P. S. et al, 1991; Boom W. R. et al, 1993) and salt-induced DNA precipitation (Miller S. A. et al, 1988) are yet other known approaches to purify nucleic acids. In a preferred embodiment, the target nucleic acid is DNA. In another embodiment, the target nucleic is RNA. Prior to applying the methods of the invention, a DNA copy (cDNA) of target RNA can be obtained using an oligonucleotide primer that hybridize to the target RNA, and extending of this primer in the presence of a reverse transcriptase and nucleoside 5′-triphosphates. The resulting DNA/RNA heteroduplex can then be rendered single-stranded using techniques known in the art, for example, denaturation at elevated temperatures. Alternatively, the RNA strand may be degraded in presence of RNase H nuclease. In certain aspects, target RNA may be used directly to initiate the target specific probe cleavage by Endonuclease V. In these aspects, Endonuclease V is preferably a mutant enzyme that can cleave DNA strand in DNA/RNA heteroduplexes. Optimally, the detection tests must be able to generate a detectable signal from samples that contain but a few copies of a nucleic acid of interest. In preferred embodiments of the invention, the detected target nucleic acid is amplified before or during the detection reaction employing one of the amplification reactions presently described in the Art.

Endonuclease V. Endonuclease V can be isolated from a variety of organisms including archaebacteria, eubacteria and eukaryotes using techniques and approaches well established in the art. For example, Endonuclease V was identified and isolated from hyperthermophiles Archaeoglobus fulgidus (Liu J. et al, 2000), Thermotoga maritima (Huang J. et al, 2001; Huang J. et al, 2002) and mice (Moe A. et al, 2003). The thermostable enzymes such as Endonucleases V from Archaeoglobus fulgidus, Thermotoga maritima are particularly useful in methods of the invention because these nucleases express activity at temperatures >50° C.

It has been well established in the Art that limited and site-directed changes in amino acid sequences of enzymes isolated from natural sources can beneficially change properties of the corresponding mutants in regards to specific applications of these proteins for scientific research and industrial purposes. For example, many DNA polymerases used today are the mutant enzymes selected to address numerous aspects of different DNA amplification and detection methods. In preferred embodiments of the invention, the Endonuclease V is a mutant Endonuclease V that, compared to the native enzyme, has improved properties for practicing the methods of the invention. Depend on the nature of the nick-directing modification (ND modification) used in manufacturing of the EndoV probes, the properties that may be improved by the Endo V enzyme mutation include but not limited to: (i) increased efficiency and cycling capabilities of the probe cleavage reaction, (ii) preferential cleavage of the probe strand when it is hybridized to the target with minimal or no cleavage of the target strand, and (iii) preferential cleavage of the probe incorporating ND modification when it is hybridized to the target compare to the cleavage of the unhybridized probe. It may be anticipated, in particular, that the detection rate and/or efficiency in methods of the invention depends on the processivity of Endonucleases V (cycling capabilities). The greater the number of EndoV probes cleaved by an Endonuclease V, the faster detection reaction proceeds. For example, Endonucleases V isolated from natural sources commonly displays elevated affinity to dI-containing duplex substrates including the cleaved substrate and, therefore, expressing limited cycling capabilities (see, e.g., Huang J. et al, 2001; Huang J. et al, 2002; Yao M. et al, 1994; Yao M. and Kow Y. W., 1994; Yao M., Kow Y. W., 1995; Yao M., Kow Y. W., 1996; Yao M., Kow Y. W., 1997). This tight-binding of the endonuclease to the cleaved duplex substrate reduces the Endonuclease V processivity or capability for cycling, i.e. when one molecule of the enzyme can cleave multiple duplex substrates. An example of this is provided by Huang J. et al, 2002 which is incorporated herein by reference. The authors prepared, isolated and studied a number of mutants of Endonuclease V from Thermotoga maritima. Several mutants, in particular, Y80A, H116A, R88A and K139A, were found to have improved cleavage cycling properties in reaction with excess of double-stranded substrate incorporating deoxyinosine modification (E:S=1:10). Similar enzyme mutation method has been described by Nelson J. R. et al, 2008 to improve the cleavage-cycling capabilities of Endonucleases V from Escherichia coli and Archaeoglobus fulgidus. Additional mutants encompassed by the present invention are described in U.S. Pat. No. 7,198,894 and WO 2008/086381 A2, which are incorporated herein by reference.

Endo-V-cleavable probes. In particular aspects of the methods, at least one endonuclease-V-cleavable oligonucleotide probe incorporates at least one nick-directing modification selected from deoxyinosine, deoxyxanthosine, deoxyuridine, abasic nucleotide and mismatched nucleotide. In other aspects, the nick-directing modification is located within three nucleotide positions from the 5′-end of the endonuclease-V-cleavable oligonucleotide probe. Those of ordinary skill in the Art will appreciate that the choice of the ND modification in design of the EndoV probes depends on the selection of a particular method of the invention, for example, post-amplification or real time detection assays. The choice of the ND modification may also depend on amounts of the target nucleic acids in the samples. For example, deoxyxanthosine, deoxyuridine, abasic nucleotide and mismatched nucleotide may be used when the target nucleic acids concentration in the samples exceeds 1, preferably 10 and even more preferably 100 nanomolar concentrations. Amounts of the target nucleic acids in the samples are commonly limited. In preferred embodiments, the detection methods are combined with the amplification of nucleic acids of interest wherein the target nucleic acids are amplified before or during the detection reaction. Use of a mismatched nucleotide as a ND modification may be advantageous in certain methods of the invention. It has been well established in the Art that Endonucleases V provide minimum or no cleavage of oligonucleotides comprising of natural nucleotides. When a mismatched nucleotide is used, the nick-directing modification appears only when the probe is hybridized to the target nucleic acid. This may lead to an exceptional target-specificity of the probe cleavage. In general, Endonucleases V may cleave any strand of the duplex incorporating a mismatched nucleotide, i.e. mismatched base pair. The preferential cleavage of the hybridized probe strand can be achieved by selection of a particular mismatched base and its neighboring match bases according to teaching provided, e.g., in Yao M., Kow Y. W., 1994, 1996 and 1997; Huang J. et al, 2001; Turner D. J. et al, 2006, which are incorporated herein by reference. Nonetheless, sufficient strand-specificity of the probe cleavage can be difficult to achieve and this may limit the use of the EndoV probes incorporating mismatched bases in real time assays.

The main activity of Endonucleases V is to recognize and cleave DNA duplexes incorporating deoxyinosine where the enzymes express maximum cleavage activity. In preferred aspects of the methods, the ND modification is deoxyinosine. Generally, the ND modification may be located anywhere within the oligonucleotide probe and preference in the location depends on the type of the ND modification. In preferred embodiments, the nick-directing modifications are located at the 5′-end of the EndoV oligonucleotide probe. For example, Endonucleases V recognize deoxyinosine and cleave the corresponding strand in both single and double-stranded DNA but exhibit approximately 4-fold preference for double-stranded over single-stranded DNA (Yao M. et al, 1994). However, the instant working example provided herein (Example 2) shows that this cleavage preference can be considerably improved up to 15-fold, and the result also underscore the importance of the deoxyinosine location within the probe. When the ND modification is deoxyinosine, it is preferably located at third, more preferably at second, and even more preferably at first nucleotide position from the 5′-end of the EndoV oligonucleotide probe.

The EndoV probes are usually selected to have sequences that are substantially complementary to a target nucleic acid sequence. The probes are designed in length and nucleotide composition to have sufficient hybridization properties to form complementary complexes with corresponding target nucleic acids. When deoxyinosine and deoxyuridine are used in the design of the probes, these ND modifications may or may not form respective Watson-Crick base pairs with cytosine and adenosine in the target nucleic acid strand. The EndoV probe sequences do not necessary need to reflect the exact sequence of the target template. For example, a non-complementary nucleotide fragment may be attached to the 3′ end of the probe, with the remainder of the probe sequence being substantially complementary to the target nucleic acid. Non-complementary bases or longer sequences can be interspersed into the probes, provided that the probe sequence has sufficient complementarity with the sequence of the targets to hybridize and thereby form substrates for the Endo V cleavage.

In certain aspects, the endonuclease-V-cleavable oligonucleotide probe incorporates at least one structural modification other than a nick-directing modification. These structural modifications can be of nucleotide and non-nucleotide nature, hydrophobic and hydrophilic, as big as natural polypeptides and as small as single atom. Examples of these structural modifications include but are not limited to chemical substances such as atoms, moieties, residues, polymers, linkers, tails, markers or nucleotide analogs, which are usually of a synthetic nature and which are not commonly present in natural nucleic acids. In a preferred embodiment, these structural modifications are duplex-stabilizing modifications. Use of such structural modifications in design of the EndoV probes of the invention may be particularly beneficial because it allows for preparing probes with elevated hybridization properties. In one embodiment, the duplex-stabilizing modifications are modified nucleotides. Examples of these modified nucleotides that are known to provide duplex stabilization include but are not limited to Locked Nucleic Acids (LNA) (Latorra D. et al, 2003a; Latorra D. et al, 2003b; Di Giusto D. A. and King G. C., 2004), Polyamide Nucleic Acids (PNA) (Egholm M. et al, 1993), ribonucleotides, 2′-O-methyl RNA and 2′-fluoro RNA, 2,6-diaminopurine and 5-methyl-cytosine nucleotides (Lebedev Y. et al, 1996), 5-propynyl-pyrimidines (Froehler B. et al, 1997), pyrazolopyrimidines or 8-aza-7-deazapurines (Petrie C. R. et al, 1998; Meyer R. B. et al, 2000; Gall A. A. et al, 2003) and different variations thereof. In another embodiment, duplex-stabilizing modifications comprise a minor groove binders (MGB) (see, e.g. Kutyavin I. V. et al, 1997; Afonina I. et al, 1997; Kutyavin I. V. et al, 1998) and intercalators (Asseline U. et al, 1984; Nguyen T. T. et al, 1989). Endonuclease V isolated from natural sources is known to have elevated binding properties to DNA duplex incorporating deoxyinosine including the cleaved products. This limits the enzyme capabilities to cycle providing multiple cleavage products. Certain structural modifications may reduce the Endo V substrate binding properties therefore promoting the cleavage cycling and accelerating the detection process. Examples of such modifications may include but not limited to ribonucleotides, 2′-O-methyl ribonucleotides, abasic sites, linkers such as polyethylene glycol linker, etc. In preferred embodiment, the structural modifications, other than the ND modifications, are introduced into the probe to improve efficiency and/or cycling capabilities of the probe cleavage reaction; preferential cleavage of the probe strand when it is hybridized to the target; preferential cleavage of the probe when it is hybridized to the target compare to the cleavage of the unhybridized probe and/or combination thereof.

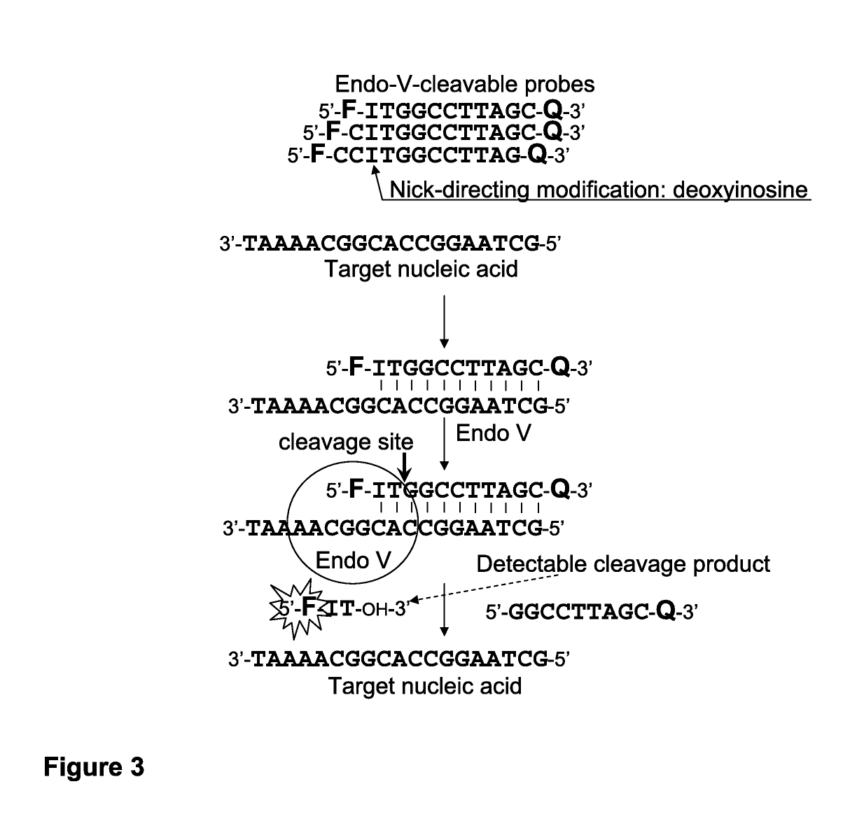

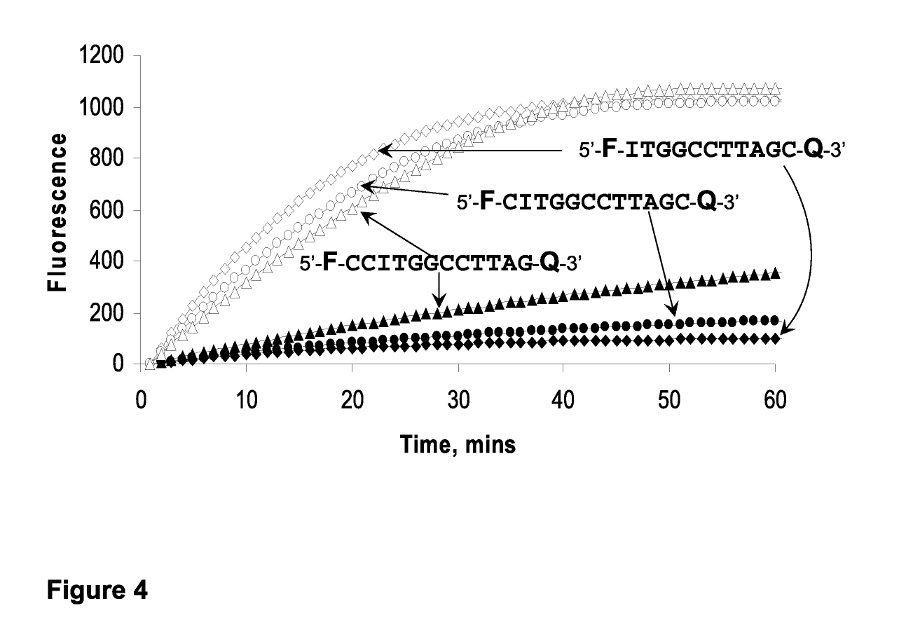

In particular aspects, the structural modification comprises a detectable label. In one embodiment, the detectable label comprises a fluorescent label. In preferred embodiment, the fluorescent label comprises two dyes that are in FRET interaction and wherein cleavage of the EndoV probe takes place between the conjugated dyes, disrupting FRET and resulting in a detectable signal. For example, the FRET dyes can be conjugated to the opposite ends of a probe as this is illustrated in FIG. 3. Cleavage of the hybridized probe at the 3′-side from deoxyinosine positioned at the 5′-end of the probe permanently and irreversibly disrupts the FRET interaction between the reporter (F) and quencher (Q) dyes and results in a detectable fluorescent signal. The instant working Example 2 and FIG. 4 illustrate use of this technology in methods of the invention for detection of target nucleic acids.

Generally, the EndoV probes are not restricted in the number, type and location of the structural modifications, other than the ND modifications, used in their design. However, those of ordinary skill in the art will appreciate that certain design rules may still apply in order to maintain the probe capability to be recognized and cleaved by Endonucleases V. For example, special attention should be paid to the modification of the probe sequences within few nucleotides on either side from the cleaved internucleotide bond and/or the nick-directing modification. Nonetheless, some modified nucleotides, especially those that support geometry of natural base pairs, may be used within these sequences, for example, 2,6-diaminopurine, 5-methyl cytosine, etc. The duplex-stabilizing tails like intercalators and minor groove binders as well as the FRET dyes should preferably be conjugated to the probe ends. On the other hand, certain structural modifications located within the few nucleotides on either side from the Endo V cleavage point and/or the nick-directing modification including the ND modification itself may benefit the detection methods of the invention by improving efficiency and/or cycling capabilities of the probe cleavage reaction, providing preferential cleavage of the probe strand when it is hybridized to the target and/or preferential cleavage of the probe when it is hybridized to the target compare to the cleavage of the unhybridized probe.

Not unlike other detection technologies, the functional efficiency of EndoV probes in methods of the invention depends, at least in part, on their hybridization properties. Depend on particular aspects, e.g. location of a ND modification within a probe: internal vs. terminal, the EndoV probes may have melting temperatures that are above, close to, or even below the detection reaction temperature. Hybridization properties of the EndoV probes are primarily defined by their length, base composition and reaction conditions (e.g. magnesium ion concentration). Duplex-stabilizing modifications can be effectively applied in the design, providing the EndoV probes with sufficient hybridization properties. In this aspect, hybridization properties of EndoV probes may be improved by amplifying target nucleic acids in the presence of base-modified duplex-stabilizing dNTPs. This technology has been described in detail (Kutyavin I. V., 2007b), which is incorporated herein by reference.

A simple estimate of the Tm value may be calculated using the equation Tm=81.5+0.41(% G+C), when a nucleic acid is in aqueous solution at 1 M NaCl. More accurate calculations can be made using the base pair thermodynamics of a “nearest-neighbors” approach (Breslauer K. J. et al, 1986; SantaLucia J. Jr., 1998). Commercial programs, including Oligo™, Primer Design and programs available on the internet, including Primer3 and Oligo Calculator can be also used to calculate a Tm of a nucleic acid sequence useful according to the invention. Commercial programs, e.g., Visual OMP (DNA software), Beacon designer 7.00 (Premier Biosoft International), may also be helpful. However, these programs are usually made for the design of PCR primers and probes, and specialty software may be used to incorporate all and/or different and numerous aspects of the invention. In a preferred embodiment, the endo-V-cleavable probes of the invention are designed using specialty computer software.

Post-amplification and real time methods of the invention: combining the EndoV probe detection with target amplification process. In many cases, the amounts of target nucleic acids in the samples are limited for direct detection. Nevertheless, the detection assays have to generate a detectable signal regardless of the amount of nucleic acids of interest in the sample. In preferred embodiment of the invention, the detected target nucleic acid is amplified before or during the detection reaction employing one of the amplification reactions presently described in the Art. Examples of the amplification reactions that may be used in methods of the invention include but not limited to PCR (e.g., Mullis K. B. et al, 1987; Mullis K. B., 1987), NASBA (e.g., Davey C. and Malek L. T., 2000; Oehlenschlager F. et al, 1996), HAD (e.g., Vincent M. et al, 2004; An L. et al., 2005), amplification methods based on the use of RNA or composite RNA/DNA primers (e.g., Cleuziat P. and Mandrand B., 1998; Kurn N., 2001; Sagawa H. et al, 2003), SDA (e.g., Walker G. T. et al, 1993; Walker G. T. et al, 1996; Fraiser M. S. et al, 1997; Walker G. T., 1998), Rolling Circle Amplification (Lizardi P., 1998 and 2001), Loop-mediated isothermal amplification (Notomi T. and Hase T., 2002), NDA (Millar D. S. et al, 2006; Van Ness J. et al, 2003a and 2003b) and other amplification reactions. Generally, there are no restrictions on choice of the amplification reaction when the Endo-V-assisted detection is performed after the amplification. As long as a particular amplification reaction provides a nucleic acid of interest at concentrations that are sufficient for the detection, the detection reaction may be performed immediately after amplification, without the isolation of amplified material, e.g., by adding the detection components, i.e. Endonuclease V and the probe, to the amplification solution and incubation the reaction at an appropriate temperature. When products of the target amplification are predominantly double-stranded, they can be rendered single stranded by any physical, chemical or biological approach before applying the detection methods. For example, double-stranded nucleic acid can be denatured at elevated temperature, e.g. 90-95° C. Certain amplification protocols can be adjusted or modified to produce the amplified target nucleic acids predominantly in a single-stranded form. For example, when the amplification reaction is PCR, it can be performed in an asymmetric format as described, e.g., in Gyllensten Ulf. B., Erlich, H. A., 1991, wherein one of the amplicons that is detected is produced at disproportionably elevated amounts compare to its complementary counterpart.

In another preferred embodiment of the invention, the target nucleic acid is amplified and detected in real time. Unlike the detection methods that are based on 5′-nuclease cleavable probes, e.g. INVEIDER™ assay, the probe cleavage by Endonuclease V may be combined with many known in the Art, including cited herein, amplification reactions to provide real time methods. These reactions, in particular, include the isothermal amplification protocols that are based on DNA strand displacement. In real time methods of the invention all amplification components such as, e.g. primers, DNA polymerase, other enzymes, etc., and detection components such as the EndoV probes and Endonuclease V are present in the same reaction mixture. This permits a target nucleic acid to be measured as the amplification reaction progresses decreasing the number of subsequent handling steps required for the detection of amplified material. Generally, the reaction composition, condition and the reaction protocol of the real time methods of the invention are determined by a particular amplification reaction. However, those of ordinary skill in the Art would appreciate that, depend on a particular amplification reaction, design of the real time methods of the invention may require certain adjustments and optimization wherein the reaction conditions such as temperature, buffering agents to maintain pH at a selected level, salts, co-factors, scavengers, and the like as well as design of all oligonucleotide components support both stages of the reaction, amplification and the detection. For example, the reaction temperatures above 48-50° C. would require use of thermostable Endonucleases V, e.g. the enzymes isolated from Archaeoglobus fulgidus and Thermotoga maritima. These and other thermostable Endonucleases V may be, in particular, useful for real time PCR methods. The EndoV probes' designs also need to address the elevated temperatures of PCR and other isothermal reactions. Magnesium salt is an important component of the methods of the invention and it presence in real time reactions is required to support enzymatic activities of DNA polymerases and Endonucleases V. An example of the reaction composition for the detection reaction is provided herein in Example 2. However, in regard to the real time methods, the concentration of magnesium may require adjustment for the optimal reaction performance. The EndoV probes are preferably made to incorporate a 3′-moiety or otherwise blocked in order to prevent the probe extension by DNA polymerases in the real time methods.

Accelerated Cascade Amplification (ACA) is based on use of multiple ND primers wherein recurring primer extension and cleavage by Endonuclease V provides rapid amplification of target nucleic acids (Nelson J. R. et al, 2008 and Kutyavin I., 2009). In particular preferred embodiment, the target nucleic acid is amplified and detected in real time using Accelerated Cascade Amplification. In these particular real time methods, the same enzyme that is Endonuclease V supports both amplification and detection reactions providing that minimum adjustment and optimization to the real time reaction composition, condition and the reaction protocol is needed.

Rate of probe cleavage by Endonuclease V is in a linear dependence to concentration of nucleic acid of interest in a sample. In particular embodiment of the invention, detection of the target nucleic acid provides for determining the amount of the target nucleic acid in or from the sample. In other embodiments, the sample comprises a plurality of different target nucleic acid sequences, wherein, with respect to each of the plurality of target nucleic acid sequences, contacting comprises contacting with at least one endo-V-cleavable oligonucleotide probe that is complementary to the respective target nucleic acid sequence, and wherein detecting comprises detecting of at least one endonuclease V-mediated cleavage product for each of the respective target-hybridized probes (multiplex detection). In this aspect, the methods of the invention can be used to detect target polymorphic variations such as, for example, sequence insertions, deletions, and other polymorphic variations as small as single nucleotide. In these cases, similar to many other detection methods known in the Art, homologous endo-V-cleavable probes can be designed and synthesized with sufficient complementarity to every target polymorphic variation to be detected. The design is made such as the probe nucleotide segments detecting polymorphic variations are located within the probes binding sites, preferably in the middle but do not incorporate the ND modification. The probes' length and sequence composition are selected such as each of the probes has sufficient hybridization properties to hybridize to a corresponding target nucleic acid providing Endo V cleavage and detectable signal at the reaction conditions when the target nucleic acid is present in the sample. Each of the probes is also designed such as it hybridize inefficiently, or ideally do not hybridize, to other nucleic acids in the sample including those representing the target polymorphic variations, other than nucleic acid of interest that the probe is designed to detect.

In particular preferred aspects, the cleavage of the hybridized probe by the endonuclease V is performed in a cycling mode providing more than one probe cleavage per a target nucleic acid molecule. The Endo V probe cleavage in a cycling mode improves sensitivity of the detection methods. The probe cleavage cycling can be promoted and stimulated by the reaction composition and conditions such as temperature, magnesium ion and salt concentration, use of mutant Endo V, structural modifications in the probes, etc. The cleavage cycling can be also stimulated and enhanced by the EndoV probe design. For example, when a ND modification is located near or at the end of the probe, the probe is designed to have its melting temperature close to the detection temperature wherein the difference between these temperatures does not exceed, e.g. 10° C. In cases of internal location of the ND modification, i.e. at the middle or nearby the middle of the probe, the EndoV probes can be designed to have elevated hybridization properties, e.g. when the probe melting temperature exceeds the detection temperature more than 10° C., because in these cases the probe cleavage products form weaker hybrids than the original, intact probe and dissociate from the target strand leaving that strand available for additional rounds of the cleavage reaction. Additional information for performing the probe cleavage reaction in a cycling mode can be found, e.g., in U.S. Pat. Nos. 5,011,769 and 5,403,711 (Duck P., Bender R., 1991; Walder J. A., Walder R. Y., 1995) which are incorporated herein by reference. The detection assays of the invention including the real time methods can incorporates one or more specialty oligonucleotides that hybridize to a target nucleic at or nearby the EndoV probe duplex. These specialty oligonucleotides may serve as enhancers of the probe cleavage reaction or help in stabilizing the probe-target duplex. In certain aspects, these specialty oligonucleotides can be provided by target amplicons that fold into a secondary structure wherein duplex of the structure is formed next to the probe-target duplex as described in Kutyavin I. V. (2007a).

Methods of the invention may be performed in both homogeneous (when all reaction components are in a solution) and heterogeneous forms (when at least one of the components is immobilized). In particular embodiments, at least one of the target nucleic acid sequence, the endo-V-cleavable oligonucleotide probe, or the endonuclease V is immobilized. Aspects of the invention also include a kit comprising at least one endo-V-cleavable oligonucleotide probe complementary to a target nucleic acid sequence. In yet another embodiment, the kit further comprises an Endonuclease V.

Methods of the invention may be performed in various reaction vessels or containers that may be made from any solid material, including but not limited to, plastic, glass, quartz, metal, etc. The reaction vessels may be of any size, wherein the reaction volume may be measured in nanoliter, microliter, milliliter or liter scales. The reaction vessels can be of any shape, e.g. tubes or plates wherein multiple reaction vessels are combined in one plate. The reaction vessels may be made from a liquid material wherein, for example, aqueous drops of the reaction mixtures of the invention are suspended and floating in oil. Methods of the invention may be performed in a micro-fluidic or fluidic card made from any material, usually plastic, and wherein the card comprises reaction chambers and channels allowing mixing the reaction components in an order or simultaneously as required by the methods of the invention.

Synthesis and design of oligonucleotide components of the invention. The oligonucleotide components of the invention such as EndoV probes, various primers including ND primers used in ACA amplification methods and other specialty oligonucleotides can be prepared by a suitable chemical synthesis method, including, for example, the phosphodiester method disclosed in Brown E. L. et al (1979), the phosphotriester method described in Narang S. A. et al (1979). The preferred approach is the diethylphosphoramidate method disclosed in Beaucage S. L., Caruthers M. H. (1981), in combination with the solid support method disclosed in Caruthers M. H., Matteucci M. D. (1984) and performed using one of commercial automated oligonucleotide synthesizer. When oligonucleotide components of the invention need to be labeled with a fluorescent dye, for example the EndoV probes, a wide range of fluorophores may be applied in designs and synthesis. Available fluorophores include but not limited to coumarin, fluorescein (FAM, usually 6-fluorescein or 6-FAM), tetrachlorofluorescein (TET), hexachloro fluorescein (HEX), rhodamine, tetramethyl rhodamine, BODIPY, Cy3, Cy5, Cy7, Texas red and ROX. Fluorophores may be chosen to absorb and emit in the visible spectrum or outside the visible spectrum, such as in the ultraviolet or infrared ranges. FRET probes of the invention commonly incorporate a pair of fluorophores, one of which may be a none-fluorescent chromophore (commonly referred as a “dark quencher”). Suitable dark quenchers described in the art include Dabcyl and its derivatives like Methyl Red. Commercial non-fluorescent quenchers, e.g., Eclipse (Glen Research) and BHQ1, BHQ2, BHQ3 (Biosearch Technologies), may be also used for synthesis of FRET probes of the invention. Preferred quenchers are either dark quenchers or fluorophores that do not fluoresce in the chosen detection range of an assay. The donor and acceptor fluorophores for manufacturing of the labeled oligonucleotide components of the invention may be selected from suitable fluorescent groups, e.g. 6-FAM (6-carboxyfluorescein); 6-hexachloro-fluorescein ([4,7,2′,4′,5′,7′-hexachloro-(3′,6′-dipivaloylfluoresceinyl)-5-carboxylic acid]); 6-tetrachloro-fluorescein ([4,7,2′,7′-tetrachloro-(3′,6′-dipivaloylfluoresceinyl)-6-carboxylic acid]); 6-TAMRA (6-carboxytetramethylrhodamine; Dabcyl (4-((4-(dimethylamino)phenyl)azo) benzoic acid); Cy5 (Indodicarbocyanine-5); Cy3 (Indo-dicarbocyanine-3); and the like. Modified nucleoside or nucleotide analogs, for example, 5-bromouracil, 5-methylcytosine, 5-iodouracil, 2-aminoadenosine (2,6-diaminopurine), 6-methyladenosine, preudouridine and the like including ND modifications like deoxyinosine and deoxyuridine, which are rarely present in natural nucleic acids may be incorporated synthetically into oligonucleotide components. The same applies to linkers, spacers, specialty tails like intercalators and minor groove binders. All these chemical components can be prepared according to methods of organic chemistry or using respective protocols that can be found in manuscripts and patents cited herein. Many structural modifications and modified nucleosides useful to prepare oligonucleotide components of the invention are available, commonly in convenient forms of phosphoramidites and specialty CPG, from commercial sources, e.g., Glen Research, Biosearch Technologies, etc.

### Detailed Exemplary Embodiments:

In detection methods that are based on target-specific probe cleavage, it is important to identify the reaction conditions and design of oligonucleotide probes wherein cleavage of a probe is accelerated in presence of a complementary target nucleic acid therefore providing for the detection of that nucleic acid of interest. The following working Examples are provided and disclosed to demonstrate certain aspects and methods of the invention for detection of target nucleic acids. The examples are provided solely for illustrative purposes, and are not intended to limit the scope of the inventive methods and applications.

### Example 1

### Materials and Methods

Synthesis of oligonucleotide components. Structures and sequences of exemplary 19-mer target oligonucleotide and EndoV FRET probes incorporating deoxyinosine are shown in FIG. 3. A 6-fluorescein reporting dye was incorporated onto the 5′-end of the probes, and a BHQ1 “dark” quencher was introduced to the 3′-end of the probes using respective phosphoramidite and CPG from Biosearch Technologies. Deoxyinosine was incorporated into the probes using dI-CE Phosphoramidite from Glen Research. Standard phosphoramidites, solid supports and reagents to perform the solid support oligonucleotide synthesis were also purchased from Glen Research. 5-Ethylthio-1H-tetrazile solution (0.25 M) was used as a coupling agent. Oligonucleotides were synthesized either on ABI394 DNA synthesizer (Applied Biosystems) or MerMaid 6 DNA synthesizer (BioAutomation Corporation) using protocols recommended by the manufacturers for 0.2 or 1 μmole synthesis scales. After the automated synthesis, oligonucleotides were deprotected in aqueous 30% ammonia solution by incubation for 2 days at room temperature, 12 hours at 55° C. or 2 hours at 70° C.

Purification of oligonucleotide components. Tri-ON oligonucleotides were purified by HPLC on a reverse phase C18 column (LUNA 5 μm, 100A, 250×4.6 mm, Phenomenex Inc) using gradient of acetonitryl in 0.1 M triethyl ammonium acetate (pH 8.0) or carbonate (pH 8.5) buffer with flow rate of 1 ml/min. A gradient profile including washing stage 0→14% (10″), 14→45% (23′), 45→90% (10″), 90→90% (5′50″), 90→0% (30″), 0→0% (7′30″) was applied for purification of all Tri-ON oligonucleotides. The product containing fractions were dried down in vacuum (SPD 1010 SpeedVac system, TermoSavant) and trityl groups were removed by treatment in 80% aqueous acetic acid at room temperature for 40-60 minutes. After addition to the detritylation reaction (100 μl) of 20 μl sodium acetate (3 M), the oligonucleotide components were precipitated in alcohol (1.5 ml), centrifuged, washed with alcohol and dried down. Concentration of the oligonucleotide components was determined based on the optical density at 260 nm and the extinction coefficients calculated for individual oligonucleotides using on-line OligoAnalyzer 3.0 software provided by Integrated DNA Technologies. Based on the measurements, convenient stock solutions in water were prepared and stored at −20° C. for further use.

Oligonucleotide quality control. The purity of all prepared oligonucleotide components was confirmed by analytical 8-20% PAAG electrophoresis, reverse phase HPLC and by spectroscopy on Cary 4000 UV-VIS spectrophotometer equipped with Cary WinUV software, Bio Package 3.0 (Varian, Inc.).

### Example 2

### An Exemplary 19-mer Target Oligonucleotide was Detected by the Cleavage of Deoxyinosine Incorporating FRET Probes in Presence of E. coli Endonuclease V

In this Example, an exemplary 19-mer target oligonucleotide was detected by the cleavage of deoxyinosine incorporating FRET probes in presence of E. coli Endonuclease V.

Reaction mixtures of 25 μl total volume were prepared on ice to incorporate the following components with indicated providers, amounts and concentrations: 19-mer target oligonucleotide at 200 nM when present in the reaction mixture; deoxyinosine incorporating FRET probes at 200 nM for each individual probe (with the target and probe structures shown in FIG. 3); E. coli Endonuclease V (New England BioLabs Inc.) at 2 nM in 50 mM KCl, 2 mM MgCl₂, 20 mM Tris-HCl (pH8.0). The reactions were incubated at 46° C., and fluorescence was monitored for 15 seconds at the end of each minute (up to 60 minutes) using SmartCycler™ (Cepheid). Initial fluorescence was subtracted.

Specifically, FIG. 3 shows a 19-mer target oligonucleotide (SEQ ID NO:4 (TAAAACGGCACCGGAATCG)) which was detected in reaction with three 11-12-mer endo-V-cleavable FRET probes incorporating deoxyinosine as a nick-directing modification located at the first (SEQ ID NO:1 (NTGGCCTTAGC)), second (SEQ ID NO:2 (CNTGGCCTTAGC)) or third (SEQ ID NO:3 (CCNTGGCCTTAG)) position from the 5′-end of the probes, respectively. Abbreviation of deoxyinosine is N in the sequence listing and I in the drawings, whereas F is 6-fluorescein and Q is BHQ1 quencher from Biosearch Technologies, Inc. The mechanism of the target detection is shown for the probe incorporating deoxyinosine at the 5′-terminal position. The example shown herein is based on fluorescence detection. Cleavage of the hybridized probe, which results in a detectable cleavage product (SEQ ID NO:5 (GGCCTTAGC)) permanently and irreversibly disrupts the FRET interaction between the reporter (F) and quencher (Q) dyes and results in a detectable fluorescent signal. Results of the fluorescence monitoring in reactions are shown in FIG. 4.

Results are shown in FIG. 4. Each real-time curve represents an average of 4 individual reactions of the same composition using the deoxyinosine-incorporating FRET probes as indicated in presence (hollow marks) and absence (black marks) of the detected 19-mer target oligonucleotide in the reaction mixture.

Specifically, FIG. 4 shows results of fluorescence monitoring of the cleavage of three deoxyinosine-incorporating FRET probes (SEQ ID NO:1 (NTGGCCTTAGC), SEQ ID NO:2 (CNTGGCCTTAGC), and SEQ ID NO:3 (CCNTGGCCTTAG)) by Endonuclease V of Escherichia coli in presence (hollow marks) and absence (black marks) of the detected 19-mer target oligonucleotide (SEQ ID NO:4 (TAAAACGGCACCGGAATCG)). Structures of the target oligonucleotide and the probes are shown in FIG. 3. The fluorescence curves are identified for each probe by arrows. A detailed description of the experimental reactions and results is provided herein under working EXAMPLE 2.

It has been established in the art (e.g. Yao M., Kow Y. W., 1995) that Endonuclease V from Escherichia coli binds very inefficiently to the oligonucleotides incorporating deoxyinosine (dI) at the first (5′-terminal) and second position from the 5′-end whether these oligonucleotides are in single-stranded or double-stranded states. The present paradigm explains the results of the FRET probe cleavage in absence of the 19-mer target oligonucleotide. For example, the 11-mer FRET probe incorporating deoxyinosine as a 5′-terminal nick-directing modification showed very inefficient Endo V cleavage (black rectangle, FIG. 4) providing a cleavage rate of 0.35±0.04%/min (percent of probe cleaved in 1 minute). The same nick-directing modification located at the second and third nucleotide from the 5′-end of the corresponding FRET probes detectably increased the probe cleavage rate to 0.45±0.03 and 0.76±0.06%/min, respectively. Surprisingly, however, the probes' cleavage was considerably accelerated in presence of the 19-mer target oligonucleotide (black marks) providing the cleavage rates of 5.36±0.35, 4.24±0.35 and 3.38±0.17%/min for probes incorporating deoxyinosine modification at first, second and third nucleotide from the 5′-end, respectively. This corresponds to 15.3, 9.4 and 4.4-fold increase in the probe cleavage when the complementary 19-mer target oligonucleotide was present in the reaction mixture. Providing such rapid target-specific cleavage, all three FRET probes were found to be completely cleaved in 50-60 minutes of the reaction (FIG. 4).

According to these data, the 11-mer FRET probe incorporating dI-modification at the 5′-terminal position is best suited for the detection of the target. It showed lowest rate of cleavage (0.35±0.04%/min) in absence of the target and conversely the highest rate of cleavage (5.36±0.35%/min) in presence of the target oligonucleotide. As appreciated in the art, presence of nucleotides at the 5′-side from the deoxyinosine modification improves Endonuclease V binding to the substrate whether it is in a single or double stranded form (Yao M., Kow Y. W., 1995). The Endo V cleavage reaction directly depends on the enzyme binding. However, the best performing FRET probe in this study has no nucleotides at the 5′-side from dI-modification, and therefore the results were unexpected. The Applicant has presently no particular theory to explain the observed phenomenon that is the decrease in the cleavage efficiency of the target-hybridized probes caused by allocation of the dI-modification from the 5′-terminal position towards the center of the probe.

### REFERENCES CITED, AND INCORPORATED HEREIN BY REFERENCE THERETO FOR THEIR RESPECTIVE TEACHINGS

Afonina I., Zivarts M., Kutyavin I., Lukhtanov E., Gamper H., Meyer R. B. (1997) Efficient Priming of PCR with Short Oligonucleotides Conjugated to a Minor Groove Binder. Nucleic Acids Res., 25: 2657-2660.
An L., Tang W., Ranalli T. A., Kim H.-J., Wytiaz J., Kong H. (2005) Characterization of a thermostable UvrD Helicase and its participation in helicase dependant amplification, J. Biol. Chem., 280, 28952-28958.
Asseline U., Delarue M., Lancelot G., Toulme F., Thuong N. T., Montenay-Garestier T., Helene C. (1984) Nucleic acid-binding molecules with high affinity and base sequence specificity: intercalating agents covalently linked to oligonucleotides. Proc. Natl. Acad. Sci. USA, 81, 3297-3301.
Ausubel F. M, Brent R., Kingston R. E., Moore D. D., Seidman J. G., Struhl K., eds., (1993) Current Protocols in Molecular Biology, Vol. 1, Chapter 2, Section I, John Wiley & Sons, New York.
Barany F., Cao W., Huang J., Lu J. (2007) Detection of nucleic acid differences using combined endonuclease cleavage and ligation reactions, U.S. Pat. No. 7,198,894.
Beaucage S. L., Caruthers M. H. (1981) Deoxynucleoside phosphoramidites—A new class of key intermediates for deoxypolynucleotide synthesis, Tetrahedron Lett., 22:1859-1862.
Boom W. R., Henriette M. A., Kievits T., Lens P. F. (1993) Process for isolating nucleic acid, U.S. Pat. No. 5,234,809.
Breslauer K. J., Frank R., Blocker H., Marky L. A. (1986) Predicting DNA duplex stability from the base sequence, Proc. Natl. Acad. Sci. USA, 83:3746-3750.
Brown E. L., Belagaje R., Ryan M. J., Khorana H. G. (1979) Chemical synthesis and cloning of a tyrosine tRNA gene, Methods Enzymol., 68:109-151.
Caruthers M. H., Matteucci M. D. (1984) Process for preparing polynucleotides, U.S. Pat. No. 4,458,066.
Cleuziat P. and Mandrand B. (1998) Method for amplifying nucleic acid sequences by strand displacement using DNA/RNA chimeric primers, U.S. Pat. No. 5,824,517.
Dahlberg J. E., Lyamichev V. I., Brow M. A. D. (1997) Detection of target nucleic acid molecules using synthesis-deficient thermostable DNA polymerase. U.S. Pat. No. 5,691,142.
Dahlberg J. E., Lyamichev V. I., Brow M. A. D. (1998) Detection of target nucleic acid molecules using thermostable 5′ nuclease. U.S. Pat. No. 5,837,450.
Davey C., Malek L. T. (2000) Nucleic acid amplification process, U.S. Pat. No. 6,063,603.
Didenko V. V. (2001) DNA probes using fluorescence resonance energy transfer (FRET): design and application. BioTechniques, 31:1106-1121.
Di Giusto D. A. and King G. C. (2004) Strong positional preference in the interaction of LNA oligonucleotides with DNA polymerase and proofreading exonuclease activities: implications for genotyping assays. Nucleic Acids Res., 32: e32.
Doty P., Marmur J. E., Schildkraut C. (1960) Strand separation and specific recombination in deoxyribonucleic acids: Physical chemical studies, Proc. Natl. Acad. Sci. USA, 46:461-476.
Duck, P., Bender, R., Crosby, W., Robertson, J. G. (1989) Nucleic acid compositions with scissile linkage useful for detecting nucleic acid sequences. U.S. Pat. No. 4,876,187.
Duck, P., Bender, R. (1991) Methods for detecting nucleic acid sequences. U.S. Pat. No. 5,011,769.
Eckstein F., ed., (1991) Oligonucleotides and Analogs: A Practical Approach. Oxford University Press, New York.
Egholm M., Buchardt O., Christensen L., Behrens C., Freier S. M., Driver D. A., Berg R. H., Kim S. K., Norden B., Nielsen P. E. (1993) PNA hybridizes to complementary oligonucleotides obeying the Watson-Crick hydrogen bonding rules, Nature, 365:566-568.
Fong W., Modrusan Z., McNevin J., Marostenmarki J., Zin B. and Bekkaoui F. (2000) Rapid solid-phase immunoassay for detection of methicillin-resistant Staphylococcus aureus using cycling probe technology. J. Clin. Microbiol., 38:2525-2529.
Förster T. (1965) Delocalized excitation and excitation transfer. In Sinanoglu, O. (ed.), Modern Quantum Chemistry, Istanbul Lectures, part III. Academic Press, New York: 93-137.
Fraiser M. S., Spargo C. A., Walker G. T., Van Cleve M., Wright D. J., Little M. C. (1997) Strand displacement amplification using thermophilic enzymes. U.S. Pat. No. 5,648,211.
Froehler B., Wagner R., Matteucci M., Jones R. J., Gutierrez A. J., Pudlo J. (1997) Enhanced triple-helix and double-helix formation with oligomers containing modified pyrimidines, U.S. Pat. No. 5,645,985.
Gait M. J., ed., (1984) Oligonucleotide Synthesis: A Practical Approach, IRL Practical Approach Series, IRL Press, Oxford.
Gall A. A., Kutyavin I. V., Vermeulen N. M. J., Dempcy R. O. (2003) Non-aggregating, non-quenching oligomers comprising nucleotide analogues; methods of synthesis and use thereof. U.S. Pat. No. 6,660,845.
Gelfand D. H., Holland P. M., Saiki R. K., Watson R. M. (1993) Homogeneous assay system using the nuclease activity of a nucleic acid polymerase. U.S. Pat. No. 5,210,015.
Gelfand D. H., Holland P. M., Saiki R. K., Watson R. M. (1996) Nucleic acid detection by the 5′-3′ exonuclease activity of polymerases acting on adjacently hybridized oligonucleotides. U.S. Pat. No. 5,487,972.
Gyllensten Ulf. B., Erlich H. A. (1991) Methods for generating single stranded DNA by the polymerase chain reaction. U.S. Pat. No. 5,066,584.
Hall J. G., Lyamichev V. I., Mast A. L., Brow M. A. D. (1999) Detection of nucleic acids by multiple invasive cleavages. U.S. Pat. No. 5,994,069.
Harvey J. J., Lee S. P., Chan K., Kim J. H., Hwang E.-S., Cha C.-Y., Knutson J. R. and Han M. K. (2004) Characterization and application of CataCleave probe in real-time detection assays, Anal. Biochem., 333:246-255.
He B., Qing H., Kow Y. W. (2000) Deoxyxanthosine in DNA is repaired by Escherichia coli endonuclease V. Mutat. Res., 459:109-114.
Higuchi R., Dollinger G., Walsh P. S., and Griffith R. (1992) Simultaneous amplification and detection of specific DNA sequences. Biotechnology, 10:413-417.
Higuchi R., Fockler C., Dollinger G., and Watson R. (1993) Kinetic PCR: Real time monitoring of DNA amplification reactions. Biotechnology, 11:1026-1030.
Huang J., Lu J., Barany F., Cao W. (2001) Multiple cleavage activities of Endonuclease V from Thermotoga maritima: Recognition and strand nicking mechanism. Biochemistry, 40, 8738-8748.
Huang J., Lu J., Barany F., and Cao W. (2002) Mutational analysis of Endonuclease V from Thermotoga maritima. Biochemistry, 41:8342-8350.
Kornberg A., and Baker T. (1992) DNA Replication, Second Edition, W. H. Freeman and Company, New York.
Kurn N. (2001) Methods and compositions for linear isothermal amplification of polynucleotide sequences, using a RNA-DNA composite primer, U.S. Pat. No. 6,251,639.
Kutyavin I. V., Lukhtanov E. A., Gamper H. B., Meyer R. B., Gall A. (1997) Oligonucleotide-cyclopropapyrroloindole conjugates as sequence specific hybridization and crosslinking agents for nucleic acids, U.S. Pat. No. 5,659,022.
Kutyavin I. V., Lukhtanov E. A., Gamper H. B., Meyer R. B. (1998) Covalently linked oligonucleotide minor groove binder conjugates, U.S. Pat. No. 5,801,155.
Kutyavin I. V., Milesi D., Belousov Y., Podyminogin M., Vorobiev A., Gorn V., Lukhtanov E. A., Vermeulen N. M. J., Mahoney W. (2006) A novel endonuclease IV post-PCR genotyping system. Nucleic Acids Res., 34: e128.
Kutyavin I. V., Milesi D., Hoekstra M. F. (2007) Abasic site endonuclease assay, U.S. Pat. No. 7,252,940.
Kutyavin I. V. (2007a) Use of products of PCR amplification carrying elements of secondary structure to improve PCR-based nucleic acid detection, WO/2007/127999.
Kutyavin I. V. (2007b) Use of base-modified deoxynucleoside triphosphates to improve nucleic acid detection, WO/2007/127992.
Kutyavin I. (2009) Accelerated cascade amplification (ACA) of nucleic acids comprising strand and sequence specific DNA nicking. US Patent application 20090047678.
Latorra D., Arar K., Hurley J. M. (2003a) Design considerations and effects of LNA in PCR primers, Mol. Cell. Probes, 17:253-259.
Latorra D., Campbell K., Wolter A., Hurley J. M. (2003b) Enhanced allele-specific PCR discrimination in SNP genotyping using 3′ locked nucleic acid (LNA) primers, Hum. Mutat., 22:79-85.
Lebedev Y., Akopyans N., Azhikina T., Shevchenko Y., Potapov V., Stecenko D., Berg D., Sverdlov E. (1996) Oligonucleotides containing 2-aminoadenine and 5-methylcytosine are more effective as primers for PCR amplification than their nonmodified counterparts, Genet. Anal., 13:15-21.
Lehninger A. L. (1975) Biochemistry, 2nd edition. New York, Worth Publishers, Inc.
Liu J., He B., Qing H., Kow Y. W. (2000) A deoxyinosine specific endonuclease from hyperthermophile, Archaeoglobus fulgidus: a homolog of Escherichia coli endonuclease V. Mut. Res., 461:169-177.
Lizardi P. (1998) Rolling circle replication reporter systems, U.S. Pat. No. 5,854,033.
Lizardi P. (2001) Rolling circle replication reporter systems. U.S. Pat. No. 6,210,884.
Mackay I. M., Arden K. E., Nitsche A. (2002) Real-time PCR in virology, Nucleic Acids Res., 30: 1292-1305.
Mackay J., Landt O. (2007) Real-time PCR fluorescent chemistries, Methods Mol. Biol., 353: 237-262.
McPherson M. J., Quirke P., Taylor G. R., Eds (1991) PCR: A Practical Approach. IRL Press, Oxford.
McPherson M. J., Quirke P., Taylor G. R., Eds (1995) PCR2: A Practical Approach. IRL Press, Oxford.
Meyer R. B., Afonina I. A., Kutyavin I. V. (2000) Oligonucleotide containing pyrazolo[3,4-d]pyrimidines for hybridization and mismatch discrimination. U.S. Pat. No. 6,127,121.
Millar D. S., Melki J. R., Grigg G. W. (2006) Isothermal strand displacement amplification using primers containing a non-regular base, WO 2006/125267.
Miller S. A., Dykes D. D. and Polesky H. F. (1988) A simple salting out procedure for extracting DNA from human nucleated cells, Nucleic Acids Res., 16:1215.
Moe A., Ringvoll J., Nordstrand L. M., Eide L., Bjørås M., Seeberg E., Rognes T., Klungland A. (2003) Incision at hypoxanthine residues in DNA by a mammalian homologue of the Escherichia coli antimutator enzyme endonuclease V. Nucleic Acids Res., 31:3893-3900.
Mullis K. B. (1987) Process for amplifying nucleic acid sequences, U.S. Pat. No. 4,683,202.
Mullis K. B., Erlich H. A., Arnheim N., Horn G. T., Saiki R. K., Scharf S. J. (1987) Process for amplifying, detecting, and/or -cloning nucleic acid sequences, U.S. Pat. No. 4,683,195.
Narang S. A., Hsiung H. M., Brousseau R. (1979) Improved phosphotriester method for the synthesis of gene fragments, Methods Enzymol., 68:90-98.
Nelson J. R., Duthie R. S., Fuller C. W., Grossmann G. A., Sekher A. (2008) Isothermal DNA amplification, PCT patent application WO/2008/086381.
Nguyen T. T., Helene C., Asseline U. (1989) Novel compounds containing an oligonucleotide sequence bounded to an intercalating agent, a process for their synthesis and their use, U.S. Pat. No. 4,835,263.
Notomi T., Hase T. (2002) Process for synthesizing nucleic acid, U.S. Pat. No. 6,410,278.
Oehlenschlager F., Schwille P. and Eigen M. (1996) Detection of HIV-1 RNA by nucleic acid sequence-based amplification combined with fluorescence correlation spectroscopy. Proc. Natl. Acad. Sci. USA, 93:12811-12816.
Petrie C. R., Meyer R. B., Tabone J. C., Hurst G. D. (1998) Cross-linking oligonucleotides. U.S. Pat. No. 5,824,796.
Robelek R., Niu L., Schmid E. L., Knoll W. (2004) Multiplexed hybridization detection of quantum dot-conjugated DNA sequences using surface plasmon enhanced fluorescence microscopy and spectrometry, Anal. Chem., 76:6160-6165.
Saba J. (2004) An Isothermal Nucleic Acid Amplification (Nick Displacement Amplification, U.S. Provisional patent application Ser. No. 60/211,975 filed on Jun. 16, 2000 and disclosed on May 16, 2004 in: http://www.wbabin.net/saba/saba13.htm.
Sagawa H., Uemori T., Mukai H., Yamamoto J., Tomono J., Kobayashi E., Enoki T., Asada K., Kato I. (2003) Method of amplifying nucleic acid. European Patent Application 1312682.
Sambrook J., Fritsch E. F. and Maniatis T. (1989) Molecular Cloning: A Laboratory Manual, 2nd Edition. Cold Spring Harbor Lab. Cold Spring Harbor, N.Y.
SantaLucia J. Jr. (1998) A unified view of polymer, dumbbell, and oligonucleotide DNA nearest-neighbor thermodynamics. Proc. Natl. Acad. Sci. USA, 95:1460-1465.
Schouten K. A. and Weiss B. (1999) Endonuclease V protects Escherichia coli against specific mutations caused by nitrous acid. Mutat. Res., 435:245-254.
Simpson D., Crosby R. M., Skopek T. R. (1988) A method for specific cloning and sequencing of human hprt cDNA for mutation analysis. Biochem. Biophys. Res. Commun., 151:487-492.
Turner D. J., Pingle M. R., Barany F. (2006) Harnessing asymmetrical substrate recognition by thermostable EndoV to achieve balanced linear amplification in multiplexed SNP typing. Biochem. Cell Biol., 84:232-242.
Van Ness J., Van Ness L. K., and Galas D. J. (2003a) Isothermal reactions for the amplification of oligonucleotides, Proc. Natl. Acad. Sci. USA, 100:4504-4509.
Van Ness J., Galas D. J., Van Ness L. K. (2003b) Exponential amplification of nucleic acids using nicking agents, US Patent Application Publication 2003/0138800.
Vincent M., Xu Y. and Kong H. (2004) Helicase dependant isothermal DNA amplification, EMBO reports, 5:795-800.
Walker G. T. (1998) Strand displacement amplification. U.S. Pat. No. 5,712,124.
Walker G. T., Linn C. P. and Nadeau J. G. (1996) DNA detection by strand displacement amplification and fluorescence polarization with signal enhancement using DNA binding protein. Nucleic Acids Res., 24:384-353.
Walker G. T., Little M. C., Nadeau J. G. (1993) Nucleic acid target generation. U.S. Pat. No. 5,270,184.
Walsh P. S., Metzger D. A. and Higuchi R. (1991) Chelex 100 as a medium for simple extraction of DNA for PCR-based typing from forensic material. Biotechniques, 10:506-513.
Walder J. A., Walder R. Y. (1995) Nucleic acid hybridization and amplification methods for detection of specific sequences in which a complementary labeled nucleic acid probe is cleaved, U.S. Pat. No. 5,403,711.
Yao M., Hatahet Z., Melamede R. J. and Kow Y. W. (1994) Purification and Characterization of a Novel Deoxyinosine-specific Enzyme, Deoxyinosine 3′-Endonuclease, from Escherichia coli. J. Biol. Chem., 269:16260-16268.
Yao M., Kow Y. W. (1994) Strand-specific Cleavage of Mismatch-containing DNA by Deoxyinosine 3′-Endonuclease from Escherichia coli. J. Biol. Chem., 269:31390-31396.
Yao M., Kow Y. W. (1995) Interaction of Deoxyinosine 3′-Endonuclease from Escherichia coli with DNA Containing Deoxyinosine. J. Biol. Chem., 270, 28609-28616.
Yao M., Kow Y. W. (1996) Cleavage of Insertion/Deletion Mismatches, Flap and Pseudo-Y DNA Structures by Deoxyinosine 3′-Endonuclease from Escherichia coli. J. Biol. Chem., 271:30672-30676.
Yao M., Kow Y. W. (1997) Further Characterization of Escherichia coli Endonuclease V. Mechanism of Recognition for Deoxyinosine, Deoxyuridine, and Base Mismatches in DNA. J. Biol. Chem., 272:30774-30779.

1. A method of detecting a target nucleic acid in a test sample, comprising:
obtaining a test sample comprising at least one target nucleic acid sequence;
contacting, in the presence of an endonuclease V, the sample with at least one endo-V-cleavable oligonucleotide probe that is complementary to the target nucleic acid sequence to provide a reaction mixture;
incubating the reaction mixture under conditions suitable to support hybridization of the at least one endo-V-cleavable oligonucleotide probe with the at least one target nucleic acid sequence and cleavage of the target-hybridized probe by the endonuclease V; and
detecting at least one endonuclease V-mediated cleavage product of the target-hybridized probe, wherein the presence of the at least one cleavage product is indicative of the presence of the target nucleic acid sequence in the test sample.

2. The method of claim 1, wherein the sample comprises a plurality of different target nucleic acid sequences, wherein, with respect to each of the plurality of target nucleic acid sequences, contacting comprises contacting with at least one endo-V-cleavable oligonucleotide probe that is complementary to the respective target nucleic acid sequence, and wherein detecting comprises detecting of at least one endonuclease V-mediated cleavage product for each of the respective target-hybridized probes.

3. The method of claim 1, wherein detecting the target nucleic acid sequence provides for determining the amount of the target nucleic acid in or from the sample.

4. The method of claim 1, wherein incubating and endonuclease V-mediated cleavage of the at least one target-hybridized probe is performed under cycling conditions suitable for providing more than one endonuclease V-mediated target-hybridized probe cleavage per target nucleic acid molecule.

5. The method of claim 1, wherein the at least one endonuclease-V-cleavable oligonucleotide probe incorporates at least one nick-directing modification selected from deoxyinosine, deoxyxanthosine, deoxyuridine, abasic nucleotide and mismatched nucleotide.

6. The method of claim 5, wherein the at least one nick-directing modification is located within three nucleotide positions from the 5′-end of the endonuclease-V-cleavable oligonucleotide probe.

7. The method of claim 6, wherein the at least one nick-directing modification is deoxyinosine located at the first, second or third nucleotide position from the 5′-end of the endo-V-cleavable oligonucleotide probe.

8. The method of claim 1, wherein the endonuclease-V-cleavable oligonucleotide probe incorporates at least one structural modification other than a nick-directing modification.

9. The method of claim 8, wherein the at least one structural modification, relative to a corresponding probe lacking the structural modification, improves at least one of: efficiency and/or cycling capabilities of the probe cleavage reaction; preferential cleavage of the probe, relative to target cleavage, when the probe is hybridized to the target sequence; preferential cleavage of the probe when it is hybridized to the target compared to cleavage of the unhybridized probe; and combinations thereof.

10. The method of claim 8, wherein the at least one structural modification comprises a duplex-stabilizing modification selected from minor groove binders, intercalators, duplex-stabilizing nucleotide analogs, and combinations thereof.

11. The method of claim 8, wherein the at least one structural modification comprises a detectable label.

12. The method of claim 11, wherein the detectable label comprises a fluorescent label.

13. The method of claim 12, wherein the fluorescent label comprises two dyes that are in FRET interaction, and wherein cleavage of the endo-V-cleavable oligonucleotide probe takes place between the dyes, disrupting FRET and resulting in a detectable signal.

14. The method of claim 1, comprising amplifying of the at least one target nucleic acid sequence before or during the incubating and/or detecting.

15. The method of claim 14, wherein amplifying comprises at least one of PCR and an isothermal reaction.

16. The method of claim 15, wherein amplifying comprises an isothermal reaction using Accelerated Cascade Amplification.

17. The method of claim 14, wherein the at least one target nucleic acid sequence is amplified and detected in real time.

18. The method of claim 1, wherein the Endonuclease V is a mutant Endonuclease V that, relative to the respective native enzyme, has improved properties in at least one of the parameters selected from: increased efficiency and/or cycling capabilities of the at least one probe cleavage reaction; preferential cleavage of the at least one probe, relative to target cleavage, when the at least one probe is hybridized to the target sequence; and preferential cleavage of the at least one probe when it is hybridized to the at least one target sequence compared to cleavage of the respective at least one unhybridized probe.

19. The method of claim 1, wherein at least one of the target nucleic acid sequence, the endo-V-cleavable oligonucleotide probe, or the endonuclease V is immobilized.

20.-38. (canceled)


SEQUENCE LISTING (5 sequences)

SEQ ID NO: 1 - DNA, 11 bp, Artificial Sequence
  11-mer endo-V-cleavable FRET probe-1
ntggccttagc

SEQ ID NO: 2 - DNA, 12 bp, artificial sequence
  12-mer endo-V-cleavable FRET probe
cntggccttagc

SEQ ID NO: 3 - DNA, 12 bp, artificial sequence
  12-mer endo-V-cleavable FRET probe
ccntggccttag

SEQ ID NO: 4 - DNA, 19 bp, artificial sequence
  19-mer target oligonucleotide
taaaacggcaccggaatcg

SEQ ID NO: 5 - DNA, 9 bp, artificial sequence
  Detectable cleavage product
ggccttagc